(19) United States
(12) Patent Application Publication
(10) Pub. No.: US 2024/0024629 A1
(43) Pub. Date: Jan. 25, 2024

(54) TORQUER FOR AN ELONGATED MEDICAL DEVICE

(71) Applicant: Corindus, Inc., Newton, MA (US)

(72) Inventors: Christopher Zirps, Sharon, MA (US); Cameron Canale, Groton, MA (US)

(21) Appl. No.: 18/256,454

(22) PCT Filed: Jan. 14, 2021
(86) PCT No.: PCT/US2021/070035

§ 371 Date: Jun. 8, 2023

(51) Int. Cl. A61M 25/01; A61M 25/09; A61B 17/12

(52) U.S. Cl. CPC A61M 25/0113; A61M 25/09; A61B 17/12118; A61M 2025/09116

(57) ABSTRACT

A torquer for an elongated medical device includes a body having a cavity defining a pathway. A first jaw is movable within the cavity. The first jaw includes a pad having a compliant property. A biasing member separate from the first jaw biases the first jaw relative to the body. An actuator movable relative to the body moves the first jaw to pinch and/or unpinch the elongated medical device with the first pad within the pathway.

### BACKGROUND

Catheters and other elongated medical devices (EMDs) may be used for minimally invasive medical procedures for the diagnosis and treatment of diseases of various vascular systems, including neurovascular intervention (NVI) also known as neurointerventional surgery, percutaneous coronary intervention (PCI) and peripheral vascular intervention (PVI). These procedures typically involve navigating a guidewire through the vasculature, and via the guidewire advancing a catheter to deliver therapy. The catheterization procedure starts by gaining access into the appropriate vessel, such as an artery or vein, with an introducer sheath using standard percutaneous techniques. Through the introducer sheath, a sheath or guide catheter is then advanced over a diagnostic guidewire to a primary location such as an internal carotid artery for NVI, a coronary ostium for PCI, or a superficial femoral artery for PVI. A guidewire suitable for the vasculature is then navigated through the sheath or guide catheter to a target location in the vasculature. In certain situations, such as in tortuous anatomy, a support catheter or microcatheter is inserted over the guidewire to assist in navigating the guidewire. The physician or operator may use an imaging system (e.g., fluoroscope) to obtain a cine with a contrast injection and select a fixed frame for use as a roadmap to navigate the guidewire or catheter to the target location, for example, a lesion. Contrast-enhanced images are also obtained while the physician delivers the guidewire or catheter so that the physician can verify that the device is moving along the correct path to the target location. While observing the anatomy using fluoroscopy, the physician manipulates the proximal end of the guidewire or catheter to direct the distal tip into the appropriate vessels toward the lesion or target anatomical location and avoid advancing into side branches.

Robotic catheter-based procedure systems have been developed that may be used to aid a physician in performing catheterization procedures such as, for example, NVI, PCI and PVI. Examples of NVI procedures include coil embolization of aneurysms, liquid embolization of arteriovenous malformations and mechanical thrombectomy of large vessel occlusions in the setting of acute ischemic stroke. In an NVI procedure, the physician uses a robotic system to gain target lesion access by controlling the manipulation of a neurovascular guidewire and microcatheter to deliver the therapy to restore normal blood flow. Target access is enabled by the sheath or guide catheter but may also require an intermediate catheter for more distal territory or to provide adequate support for the microcatheter and guidewire. The distal tip of a guidewire is navigated into, or past, the lesion depending on the type of lesion and treatment. For treating aneurysms, the microcatheter is advanced into the lesion and the guidewire is removed and several embolization coils are deployed into the aneurysm through the microcatheter and used to block blood flow into the aneurysm. For treating arteriovenous malformations, a liquid embolic is injected into the malformation via a microcatheter. Mechanical thrombectomy to treat vessel occlusions can be achieved either through aspiration and/or use of a stent retriever. Depending on the location of the clot, aspiration is either done through an aspiration catheter, or through a microcatheter for smaller arteries. Once the aspiration catheter is at the lesion, negative pressure is applied to remove the clot through the catheter. Alternatively, the clot can be removed by deploying a stent retriever through the microcatheter. Once the clot has integrated into the stent retriever, the clot is retrieved by retracting the stent retriever and microcatheter (or intermediate catheter) into the guide catheter.

In PCI, the physician uses a robotic system to gain lesion access by manipulating a coronary guidewire to deliver the therapy and restore normal blood flow. The access is enabled by seating a guide catheter in a coronary ostium. The distal tip of the guidewire is navigated past the lesion and, for complex anatomies, a microcatheter may be used to provide adequate support for the guidewire. The blood flow is restored by delivering and deploying a stent or balloon at the lesion. The lesion may need preparation prior to stenting, by either delivering a balloon for pre-dilation of the lesion, or by performing atherectomy using, for example, a laser or rotational atherectomy catheter and a balloon over the guidewire. Diagnostic imaging and physiological measurements may be performed to determine appropriate therapy by using imaging catheters or fractional flow reserve (FFR) measurements.

In PVI, the physician uses a robotic system to deliver the therapy and restore blood flow with techniques similar to NVI. The distal tip of the guidewire is navigated past the lesion and a microcatheter may be used to provide adequate support for the guidewire for complex anatomies. The blood flow is restored by delivering and deploying a stent or balloon to the lesion. As with PCI, lesion preparation and diagnostic imaging may be used as well.

When support at the distal end of a catheter or guidewire is needed, for example, to navigate tortuous or calcified vasculature, to reach distal anatomical locations, or to cross hard lesions, an over-the-wire (OTW) catheter or coaxial system is used. An OTW catheter has a lumen for the guidewire that extends the full length of the catheter. This provides a relatively stable system because the guidewire is supported along the whole length. This system, however, has some disadvantages, including higher friction, and longer overall length compared to rapid-exchange catheters (see below). Typically to remove or exchange an OTW catheter while maintaining the position of the indwelling guidewire, the exposed length (outside of the patient) of guidewire must be longer than the OTW catheter. A 300 cm long guidewire is typically sufficient for this purpose and is often referred to as an exchange length guidewire. Due to the length of the guidewire, two operators are needed to remove or exchange an OTW catheter. This becomes even more challenging if a triple coaxial, known in the art as a tri-axial system, is used (quadruple coaxial catheters have also been known to be used). However, due to its stability, an OTW system is often used in NVI and PVI procedures. On the other hand, PCI procedures often use rapid exchange (or monorail) catheters. The guidewire lumen in a rapid exchange catheter runs only through a distal section of the catheter, called the monorail or rapid exchange (RX) section. With a RX system, the operator manipulates the interventional devices parallel to each other (as opposed to with an OTW system, in which the devices are manipulated in a serial configuration), and the exposed length of guidewire only needs to be slightly longer than the RX section of the catheter. A rapid exchange length guidewire is typically 180-200 cm long. Given the shorter length guidewire and monorail, RX catheters can be exchanged by a single operator. However, RX catheters are often inadequate when more distal support is needed.

During procedures a variety of wire-like devices, such as guidewires, stent retrievers, and coils, are gripped by their shaft to linearly and/or rotationally manipulate the devices in the patient anatomy. EMDs are typically gripped by the operator's fingers or with a pin vice like device, commonly referred to as a torquer device.

A torquer is used by an operator to releasably pinch and unpinch a portion of an EMD, such as a guidewire, during a procedure. The torquer is used to releasably fix a portion of an EMD to allow a user to manipulate the EMD by rotating and/or translating the EMD.

The diameter of devices used in procedures vary from 0.009-0.038 inch (0.229-0.965 mm) outer diameter (OD). Commercially available torquers are typically designed for a specific OD device. For example, one torquer would be used to manipulate a 0.014 inch (0.356 mm) OD device and a different torquer would be used to manipulate a 0.038 inch (0.965 mm) OD device.

### SUMMARY

A torquer for an elongated medical device includes a body having a cavity defining a pathway. A first jaw is movable within the cavity. The first jaw includes a pad having a compliant property. A biasing member separate from the first jaw biases the first jaw relative to the body. An actuator movable relative to the body moves the first jaw to pinch and/or unpinch the elongated medical device with the first pad within the pathway. In one embodiment the pad having a compliant property is formed from an elastomeric material. In one embodiment the actuator is a knob.

In one embodiment a torquer releasably engaging an elongated medical device, includes a body having a cavity defining a pathway. At least two jaws are movable within the cavity, each jaw having a pad base and a pad secured thereto, wherein the jaws are not connected to one another. A biasing member separate from the jaws biases the jaws relative to the body. A knob movable relative to the body moves the jaws relative to one another pinching or unpinching the elongated medical device with the pads within the pathway.

In one embodiment a torquer releasably engaging an elongated medical device, includes a body having a cavity defining a pathway. At least two jaws move within the cavity, each jaw having an elastomeric pad, wherein the jaws are not connected to one another. A biasing member separate from the jaws biases the jaws relative to the body. A knob movable relative to the body moves the jaws relative to one another to pinch or unpinch the elongated medical device with the elastomeric pads within the pathway, wherein the pressure between the elastomeric pads and the elongated medical device is substantially equalized along an entire length of the elastomeric pads in a fully pinched position.

### BRIEF DESCRIPTION OF THE DRAWINGS

FIG. 1 is a schematic view of an exemplary catheter-based procedure system in accordance with an embodiment.

FIG. 2 is a schematic block diagram of an exemplary catheter-based procedure system in accordance with an embodiment.

FIG. 3 is an isometric view of an exemplary bedside system of a catheter-based procedure system in accordance with an embodiment.

FIG. 4 is an isometric view of a passive torquer assembly with pads.

FIG. 5 is an exploded view of the passive torquer assembly of FIG. 4.

FIG. 6 is an exploded view of the jaws of the passive torquer assembly of FIG. 4.

FIG. 7 is a cross-sectional view taken generally in the X-Z plane of FIG. 4 showing the torquer assembly of FIG. 4 with the pads in the engaged position with a guidewire (EMD).

FIG. 8 is a cross-sectional view (not to scale) taken generally in the X-Z plane of FIG. 4 showing the torquer assembly of FIG. 4 with the pads in a non-aligned position in the process of disengagement with a guidewire (EMD).

FIG. 9 is a cross-sectional view taken generally in the X-Z plane of FIG. 4 showing the torquer assembly of FIG. 4 with the pads in the disengaged position.

FIG. 10 is a cross-sectional view (not to scale) taken generally in the X-Z plane of FIG. 4 showing an embodiment of the torquer assembly of FIG. 4 with a single movable jaw in the process of disengagement with a guidewire (EMD).

FIG. 11 is an isometric view an active torquer assembly with pads.

FIG. 12 is an exploded view showing some components of the active torquer assembly of FIG. 11.

FIG. 13 is an exploded view showing internal components of the active torquer assembly of FIG. 11.

FIG. 14 is an exploded view of the jaws of the active torquer assembly of FIG. 11.

FIG. 15 is a cross-sectional view taken generally in the X-Z plane of FIG. 11 showing the torquer assembly of FIG. 11 with the knob unthreaded and the pads in the disengaged position.

FIG. 16 is a cross-sectional view taken generally in the X-Z plane of FIG. 11 showing the torquer assembly of FIG. 11 with the knob threaded and the pads in the disengaged position.

FIG. 17 is a cross-sectional view taken generally in the X-Z plane of FIG. 11 showing the torquer assembly of FIG. 11 with the pads in the engaged position.

FIG. 18 is a view of the passive torquer assembly of FIG. 4 in a device module.

### DETAILED DESCRIPTION OF THE EXAMPLE EMBODIMENTS

### Definitions

The term elongated medical device (EMD) refers to, but is not limited to, catheters (e.g., guide catheters, microcatheters, balloon/stent catheters), wire-based devices (e.g., guidewires, embolization coils, stent retrievers, etc.), and medical devices comprising any combination of these. Wire-based EMDs include but are not limited to guidewires, microwires, a proximal pusher for embolization coils, stent retrievers, self-expanding stents, and flow divertors. Typically wire-based EMDs do not have a hub or handle at its proximal terminal end. In one embodiment the EMD is a catheter having a hub at a proximal end of the catheter and a flexible shaft extending from the hub toward the distal end of the catheter, wherein the shaft is more flexible than the hub. In one embodiment the catheter includes an intermediary portion that transitions between the hub and the shaft that has an intermediate flexibility that is less rigid than the hub and more rigid than the shaft. In one embodiment the intermediary portion is a strain relief.

The terms distal and proximal define relative locations of two different features. With respect to a robotic drive the terms distal and proximal are defined by the position of the robotic drive in its intended use relative to a patient. When used to define a relative position, the distal feature is the feature of the robotic drive that is closer to the patient than a proximal feature when the robotic drive is in its intended in-use position. Within a patient, any vasculature landmark further away along the path from the access point is considered more distal than a landmark closer to the access point, where the access point is the point at which the EMD enters the patient. Similarly, the proximal feature is the feature that is farther from the patient than the distal feature when the robotic drive in its intended in-use position. When used to define direction, the distal direction refers to a path on which something is moving or is aimed to move or along which something is pointing or facing from a proximal feature toward a distal feature and/or patient when the robotic drive is in its intended in-use position. The proximal direction is the opposite direction of the distal direction.

The term longitudinal axis of a member (for example, an EMD or other element in the catheter-based procedure system) is the line or axis along the length of the member that passes through the center of the transverse cross section of the member in the direction from a proximal portion of the member to a distal portion of the member. For example, the longitudinal axis of a guidewire is the central axis in the direction from a proximal portion of the guidewire toward a distal portion of the guidewire even though the guidewire may be non-linear in the relevant portion.

The terms top, up, and upper refer to the general direction away from the direction of gravity and the terms bottom, down, and lower refer to the general direction in the direction of gravity.

The term axial movement of a member refers to translation of the member along the longitudinal axis of the member.

The term rotational movement of a member refers to the change in angular orientation of the member about the local longitudinal axis of the member.

The term axial insertion refers to inserting a first member into a second member along the longitudinal axis of the second member.

The term force refers to an agent which causes or tends to cause motion of a body. A force acting on a body may change the motion of the body, retard the motion of the body, balance the forces already acting on the body, and give rise to internal stresses in the body.

The term torque refers to an agent which causes or tends to cause rotational motion of a physical body. A torque acting on a body may change the rotational motion of the body, retard the rotational motion of the body, balance the torques already acting on the body, and give rise to internal stresses in the body.

The term fixed means no intentional relative movement of a first member with respect to a second member during operation.

The term pinch refers to releasably fixing an EMD to a member such that the EMD and member move together when the member moves. Rotational movement of the member will result in rotational movement of the EMD in the pinched condition. The term unpinch refers to releasing the EMD from a member such that the EMD and member move independently when the member moves. In an unpinched condition the EMD can be moved/rotated relative to the member.

The term collet refers to a device that can releasably fix a portion of an EMD. The term fixed here means no intentional relative movement of the collet and EMD during operation.

The term torquer refers to a device that releasably pinches and unpinches a portion of an EMD, such as a guidewire. The term torquer is a generally accepted term used by medical professionals in catheter procedures to indicate a device used to rotate an EMD and/or translate an EMD. A torquer is also known generally as a collet or pin-vice. Torquers described herein are used ex vivo to pinch a portion of an EMD outside of the patient's body.

### Description of Embodiments

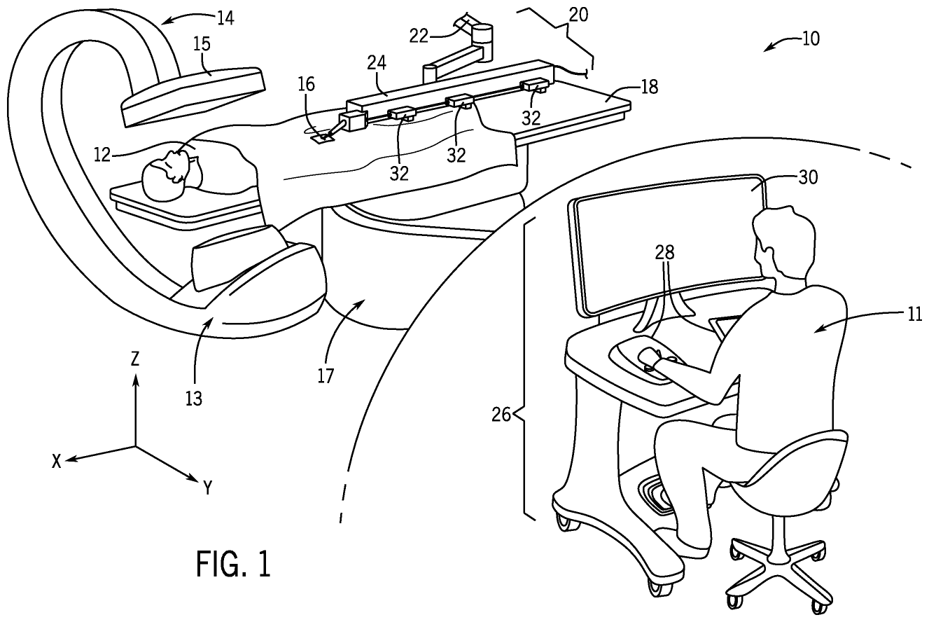

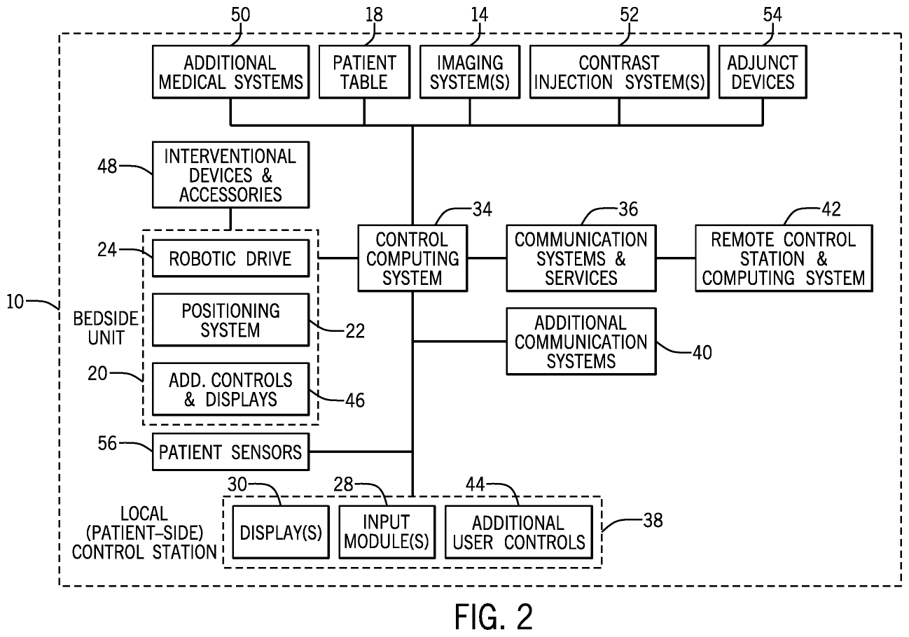

FIG. 1 is a perspective view of an exemplary catheter-based procedure system 10 in accordance with an embodiment. Catheter-based procedure system 10 may be used to perform catheter-based medical procedures, e.g., percutaneous intervention procedures such as a percutaneous coronary intervention (PCI) (e.g., to treat STEMI), a neurovascular interventional procedure (NVI) (e.g., to treat an emergent large vessel occlusion (ELVO)), peripheral vascular intervention procedures (PVI) (e.g., for critical limb ischemia (CLI), etc.). Catheter-based medical procedures may include diagnostic catheterization procedures during which one or more catheters or other elongated medical devices (EMDs) are used to aid in the diagnosis of a patient's disease. For example, during one embodiment of a catheter-based diagnostic procedure, a contrast media is injected onto one or more arteries through a catheter and an image of the patient's vasculature is taken. Catheter-based medical procedures may also include catheter-based therapeutic procedures (e.g., angioplasty, stent placement, treatment of peripheral vascular disease, clot removal, arterial venous malformation therapy, treatment of aneurysm, etc.) during which a catheter (or other EMD) is used to treat a disease. Therapeutic procedures may be enhanced by the inclusion of adjunct devices 54 (shown in FIG. 2) such as, for example, intravascular ultrasound (IVUS), optical coherence tomography (OCT), fractional flow reserve (FFR), etc. It should be noted, however, that one skilled in the art would recognize that certain specific percutaneous intervention devices or components (e.g., type of guidewire, type of catheter, etc.) may be selected based on the type of procedure that is to be performed. Catheter-based procedure system 10 can perform any number of catheter-based medical procedures with minor adjustments to accommodate the specific percutaneous intervention devices to be used in the procedure.

Catheter-based procedure system 10 includes, among other elements, a bedside unit 20 and a control station 26. Bedside unit 20 includes a robotic drive 24 and a positioning system 22 that are located adjacent to a patient 12. Patient 12 is supported on a patient table 18. The positioning system 22 is used to position and support the robotic drive 24. The positioning system 22 may be, for example, a robotic arm, an articulated arm, a holder, etc. The positioning system 22 may be attached at one end to, for example, a rail on the patient table 18, a base, or a cart. The other end of the positioning system 22 is attached to the robotic drive 24. The positioning system 22 may be moved out of the way (along with the robotic drive 24) to allow for the patient 12 to be placed on the patient table 18. Once the patient 12 is positioned on the patient table 18, the positioning system 22 may be used to situate or position the robotic drive 24 relative to the patient 12 for the procedure. In an embodiment, patient table 18 is operably supported by a pedestal 17, which is secured to the floor and/or earth. Patient table 18 is able to move with multiple degrees of freedom, for example, roll, pitch, and yaw, relative to the pedestal 17. Bedside unit 20 may also include controls and displays 46 (shown in FIG. 2). For example, controls and displays may be located on a housing of the robotic drive 24.

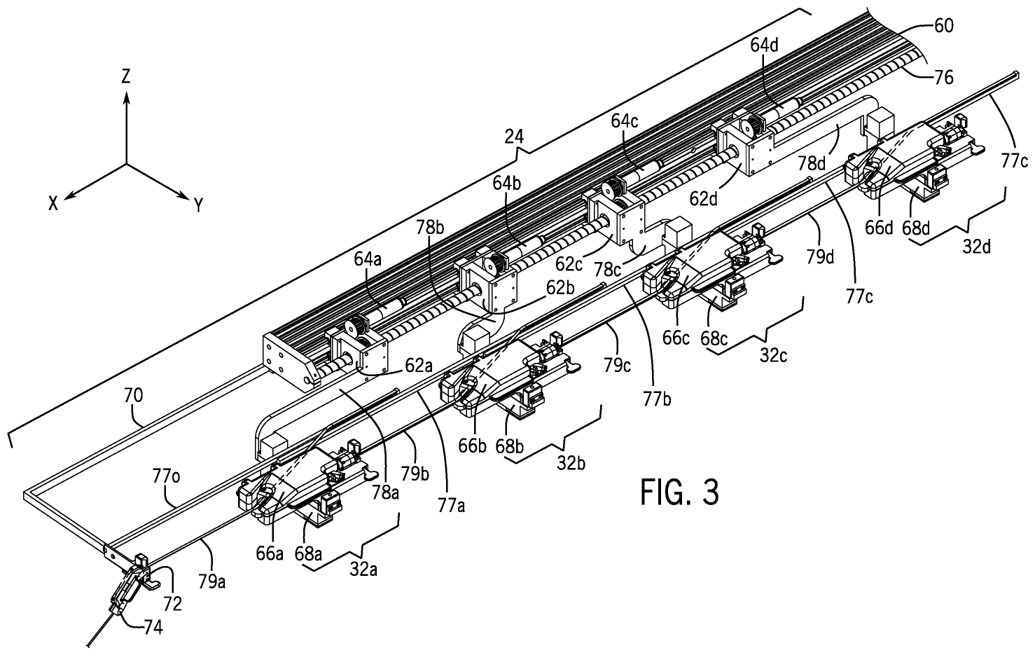

Generally, the robotic drive 24 may be equipped with the appropriate percutaneous interventional devices and accessories 48 (shown in FIG. 2) (e.g., guidewires, various types of catheters including balloon catheters, stent delivery systems, stent retrievers, embolization coils, liquid embolics, aspiration pumps, device to deliver contrast media, medicine, hemostasis valve adapters, syringes, stopcocks, inflation device, etc.) to allow the user or operator 11 to perform a catheter-based medical procedure via a robotic system by operating various controls such as the controls and inputs located at the control station 26. Bedside unit 20, and in particular robotic drive 24, may include any number and/or combination of components to provide bedside unit 20 with the functionality described herein. A user or operator 11 at control station 26 is referred to as the control station user or control station operator and referred to herein as user or operator. A user or operator at bedside unit 20 is referred to as bedside unit user or bedside unit operator. The robotic drive 24 includes a plurality of device modules 32a-d mounted to a rail or linear member 60 (shown in FIG. 3). The rail or linear member 60 guides and supports the device modules. Each of the device modules 32a-d may be used to drive an EMD such as a catheter or guidewire. For example, the robotic drive 24 may be used to automatically feed a guidewire into a diagnostic catheter and into a guide catheter in an artery of the patient 12. One or more devices, such as an EMD, enter the body (e.g., a vessel) of the patient 12 at an insertion point 16 via, for example, an introducer sheath.

Bedside unit 20 is in communication with control station 26, allowing signals generated by the user inputs of control station 26 to be transmitted wirelessly or via hardwire to bedside unit 20 to control various functions of bedside unit 20. As discussed below, control station 26 may include a control computing system 34 (shown in FIG. 2) or be coupled to the bedside unit 20 through a control computing system 34. Bedside unit 20 may also provide feedback signals (e.g., loads, speeds, operating conditions, warning signals, error codes, etc.) to control station 26, control computing system 34 (shown in FIG. 2), or both. Communication between the control computing system 34 and various components of the catheter-based procedure system 10 may be provided via a communication link that may be a wireless connection, cable connections, or any other means capable of allowing communication to occur between components. Control station 26 or other similar control system may be located either at a local site (e.g., local control station 38 shown in FIG. 2) or at a remote site (e.g., remote control station and computer system 42 shown in FIG. 2). Catheter procedure system 10 may be operated by a control station at the local site, a control station at a remote site, or both the local control station and the remote control station at the same time. At a local site, user or operator 11 and control station 26 are located in the same room or an adjacent room to the patient 12 and bedside unit 20. As used herein, a local site is the location of the bedside unit 20 and a patient 12 or subject (e.g., animal or cadaver) and the remote site is the location of a user or operator 11 and a control station 26 used to control the bedside unit 20 remotely. A control station 26 (and a control computing system) at a remote site and the bedside unit 20 and/or a control computing system at a local site may be in communication using communication systems and services 36 (shown in FIG. 2), for example, through the Internet. In an embodiment, the remote site and the local (patient) site are away from one another, for example, in different rooms in the same building, different buildings in the same city, different cities, or other different locations where the remote site does not have physical access to the bedside unit 20 and/or patient 12 at the local site.

Control station 26 generally includes one or more input modules 28 configured to receive user inputs to operate various components or systems of catheter-based procedure system 10. In the embodiment shown, control station 26 allows the user or operator 11 to control bedside unit 20 to perform a catheter-based medical procedure. For example, input modules 28 may be configured to cause bedside unit 20 to perform various tasks using percutaneous intervention devices (e.g., EMDs) interfaced with the robotic drive 24 (e.g., to advance, retract, or rotate a guidewire, advance, retract or rotate a catheter, inflate or deflate a balloon located on a catheter, position and/or deploy a stent, position and/or deploy a stent retriever, position and/or deploy a coil, inject contrast media into a catheter, inject liquid embolics into a catheter, inject medicine or saline into a catheter, aspirate on a catheter, or to perform any other function that may be performed as part of a catheter-based medical procedure). Robotic drive 24 includes various drive mechanisms to cause movement (e.g., axial and rotational movement) of the components of the bedside unit 20 including the percutaneous intervention devices.

In one embodiment, input modules 28 may include one or more touch screens, joysticks, scroll wheels, and/or buttons. In addition to input modules 28, the control station 26 may use additional user controls 44 (shown in FIG. 2) such as foot switches and microphones for voice commands, etc. Input modules 28 may be configured to advance, retract, or rotate various components and percutaneous intervention devices such as, for example, a guidewire, and one or more catheters or microcatheters. Buttons may include, for example, an emergency stop button, a multiplier button, device selection buttons and automated move buttons. When an emergency stop button is pushed, the power (e.g., electrical power) is shut off or removed to bedside unit 20. When in a speed control mode, a multiplier button acts to increase or decrease the speed at which the associated component is moved in response to a manipulation of input modules 28. When in a position control mode, a multiplier button changes the mapping between input distance and the output commanded distance. Device selection buttons allow the user or operator 11 to select which of the percutaneous intervention devices loaded into the robotic drive 24 are controlled by input modules 28. Automated move buttons are used to enable algorithmic movements that the catheter-based procedure system 10 may perform on a percutaneous intervention device without direct command from the user or operator 11. In one embodiment, input modules 28 may include one or more controls or icons (not shown) displayed on a touch screen (that may or may not be part of a display 30), that, when activated, causes operation of a component of the catheter-based procedure system 10. Input modules 28 may also include a balloon or stent control that is configured to inflate or deflate a balloon and/or deploy a stent. Each of the input modules 28 may include one or more buttons, scroll wheels, joysticks, touch screen, etc. that may be used to control the particular component or components to which the control is dedicated. In addition, one or more touch screens may display one or more icons (not shown) related to various portions of input modules 28 or to various components of catheter-based procedure system 10.

Control station 26 may include a display 30. In other embodiments, the control station 26 may include two or more displays 30. Display 30 may be configured to display information or patient specific data to the user or operator 11 located at control station 26. For example, display 30 may be configured to display image data (e.g., X-ray images, MRI images, CT images, ultrasound images, etc.), hemodynamic data (e.g., blood pressure, heart rate, etc.), patient record information (e.g., medical history, age, weight, etc.), lesion or treatment assessment data (e.g., IVUS, OCT, FFR, etc.). In addition, display 30 may be configured to display procedure specific information (e.g., procedural checklist, recommendations, duration of procedure, catheter or guidewire position, volume of medicine or contrast agent delivered, etc.). Further, display 30 may be configured to display information to provide the functionalities associated with control computing system 34 (shown in FIG. 2). Display 30 may include touch screen capabilities to provide some of the user input capabilities of the system.

Catheter-based procedure system 10 also includes an imaging system 14. Imaging system 14 may be any medical imaging system that may be used in conjunction with a catheter based medical procedure (e.g., non-digital X-ray, digital X-ray, CT, MRI, ultrasound, etc.). In an exemplary embodiment, imaging system 14 is a digital X-ray imaging device that is in communication with control station 26. In one embodiment, imaging system 14 may include a C-arm (shown in FIG. 1) that allows imaging system 14 to partially or completely rotate around patient 12 in order to obtain images at different angular positions relative to patient 12 (e.g., sagittal views, caudal views, anterior-posterior views, etc.). In one embodiment imaging system 14 is a fluoroscopy system including a C-arm having an X-ray source 13 and a detector 15, also known as an image intensifier.

Imaging system 14 may be configured to take X-ray images of the appropriate area of patient 12 during a procedure. For example, imaging system 14 may be configured to take one or more X-ray images of the head to diagnose a neurovascular condition. Imaging system 14 may also be configured to take one or more X-ray images (e.g., real time images) during a catheter-based medical procedure to assist the user or operator 11 of control station 26 to properly position a guidewire, guide catheter, microcatheter, stent retriever, coil, stent, balloon, etc. during the procedure. The image or images may be displayed on display 30. For example, images may be displayed on display 30 to allow the user or operator 11 to accurately move a guide catheter or guidewire into the proper position.

In order to clarify directions, a rectangular coordinate system is introduced with X, Y, and Z axes. The positive X axis is oriented in a longitudinal (axial) distal direction, that is, in the direction from the proximal end to the distal end, stated another way from the proximal to distal direction. The Y and Z axes are in a transverse plane to the X axis, with the positive Z axis oriented up, that is, in the direction opposite of gravity, and the Y axis is automatically determined by right-hand rule.

FIG. 2 is a block diagram of catheter-based procedure system 10 in accordance with an exemplary embodiment. Catheter-procedure system 10 may include a control computing system 34. Control computing system 34 may physically be, for example, part of control station 26 (shown in FIG. 1). Control computing system 34 may generally be an electronic control unit suitable to provide catheter-based procedure system 10 with the various functionalities described herein. For example, control computing system 34 may be an embedded system, a dedicated circuit, a general-purpose system programmed with the functionality described herein, etc. Control computing system 34 is in communication with bedside unit 20, communications systems and services 36 (e.g., Internet, firewalls, cloud services, session managers, a hospital network, etc.), a local control station 38, additional communications systems 40 (e.g., a telepresence system), a remote control station and computing system 42, and patient sensors 56 (e.g., electrocardiogram (ECG) devices, electroencephalogram (EEG) devices, blood pressure monitors, temperature monitors, heart rate monitors, respiratory monitors, etc.). The control computing system is also in communication with imaging system 14, patient table 18, additional medical systems 50, contrast injection systems 52 and adjunct devices 54 (e.g., IVUS, OCT, FFR, etc.). The bedside unit 20 includes a robotic drive 24, a positioning system 22 and may include additional controls and displays 46. As mentioned above, the additional controls and displays may be located on a housing of the robotic drive 24. Interventional devices and accessories 48 (e.g., guidewires, catheters, etc.) interface to the bedside system 20. In an embodiment, interventional devices and accessories 48 may include specialized devices (e.g., IVUS catheter, OCT catheter, FFR wire, diagnostic catheter for contrast, etc.) which interface to their respective adjunct devices 54, namely, an IVUS system, an OCT system, and FFR system, etc.

In various embodiments, control computing system 34 is configured to generate control signals based on the user's interaction with input modules 28 (e.g., of a control station 26 (shown in FIG. 1) such as a local control station 38 or a remote control station 42) and/or based on information accessible to control computing system 34 such that a medical procedure may be performed using catheter-based procedure system 10. The local control station 38 includes one or more displays 30, one or more input modules 28, and additional user controls 44. The remote control station and computing system 42 may include similar components to the local control station 38. The remote 42 and local 38 control stations can be different and tailored based on their required functionalities. The additional user controls 44 may include, for example, one or more foot input controls. The foot input control may be configured to allow the user to select functions of the imaging system 14 such as turning on and off the X-ray and scrolling through different stored images. In another embodiment, a foot input device may be configured to allow the user to select which devices are mapped to scroll wheels included in input modules 28. Additional communication systems 40 (e.g., audio conference, video conference, telepresence, etc.) may be employed to help the operator interact with the patient, medical staff (e.g., angio-suite staff), and/or equipment in the vicinity of the bedside.

Catheter-based procedure system 10 may be connected or configured to include any other systems and/or devices not explicitly shown. For example, catheter-based procedure system 10 may include image processing engines, data storage and archive systems, automatic balloon and/or stent inflation systems, medicine injection systems, medicine tracking and/or logging systems, user logs, encryption systems, systems to restrict access or use of catheter-based procedure system 10, etc.

As mentioned, control computing system 34 is in communication with bedside unit 20 which includes a robotic drive 24, a positioning system 22 and may include additional controls and displays 46, and may provide control signals to the bedside unit 20 to control the operation of the motors and drive mechanisms used to drive the percutaneous intervention devices (e.g., guidewire, catheter, etc.). The various drive mechanisms may be provided as part of a robotic drive 24.

FIG. 3 is a perspective view of a robotic drive for a catheter-based procedure system 10 in accordance with an embodiment. In FIG. 3, a robotic drive 24 includes multiple device modules 32a-d coupled to a linear member 60. Each device module 32a-d is coupled to the linear member 60 via a stage 62a-d moveably mounted to the linear member 60. A device module 32a-d may be connected to a stage 62a-d using a connector such as an offset bracket 78a-d. In another embodiment, the device module 32a-d is directly mounted to the stage 62a-d. Each stage 62a-d may be independently actuated to move linearly along the linear member 60. Accordingly, each stage 62a-d (and the corresponding device module 32a-d coupled to the stage 62a-d) may independently move relative to each other and the linear member 60. A drive mechanism is used to actuate each stage 62a-d. In the embodiment shown in FIG. 3, the drive mechanism includes independent stage translation motors 64a-d coupled to each stage 62a-d and a stage drive mechanism 76, for example, a lead screw via a rotating nut, a rack via a pinion, a belt via a pinion or pulley, a chain via a sprocket, or the stage translation motors 64a-d may be linear motors themselves. In some embodiments, the stage drive mechanism 76 may be a combination of these mechanisms, for example, each stage 62a-d could employ a different type of stage drive mechanism. In an embodiment where the stage drive mechanism is a lead screw and rotating nut, the lead screw may be rotated and each stage 62a-d may engage and disengage from the lead screw to move, e.g., to advance or retract. In the embodiment shown in FIG. 3, the stages 62a-d and device modules 32a-d are in a serial drive configuration.

Each device module 32a-d includes a drive module 68a-d and a cassette 66a-d mounted on and coupled to the drive module 68a-d. In the embodiment shown in FIG. 3, each cassette 66a-d is mounted to the drive module 68a-d in a vertical orientation. In other embodiments, each cassette 66a-d may be mounted to the drive module 68a-d in other mounting orientations. Each cassette 66a-d is configured to interface with and support a proximal portion of an EMD (not shown). In addition, each cassette 66a-d may include elements to provide one or more degrees of freedom in addition to the linear motion provided by the actuation of the corresponding stage 62a-d to move linearly along the linear member 60. For example, the cassette 66a-d may include elements that may be used to rotate the EMD when the cassette is coupled to the drive module 68a-d. Each drive module 68a-d includes at least one coupler to provide a drive interface to the mechanisms in each cassette 66a-d to provide the additional degree of freedom. Each cassette 66a-d also includes a channel in which a device support 79a-d is positioned, and each device support 79a-d is used to prevent an EMD from buckling. A support arm 77a, 77b, and 77c is attached to each device module 32a, 32b, and 32c, respectively, to provide a fixed point for support of a proximal end of the device supports 79b, 79c, and 79d, respectively. The robotic drive 24 may also include a device support connection 72 connected to a device support 79, a distal support arm 70 and a support arm 77o. Support arm 77o is used to provide a fixed point for support of the proximal end of the distal most device support 79a housed in the distal most device module 32a. In addition, an introducer interface support (redirector) 74 may be connected to the device support connection 72 and an EMD (e.g., an introducer sheath). The configuration of robotic drive 24 has the benefit of reducing volume and weight of the drive robotic drive 24 by using actuators on a single linear member.

To prevent contaminating the patient with pathogens, healthcare staff use aseptic technique in a room housing the bedside unit 20 and the patient 12 or subject (shown in FIG. 1). A room housing the bedside unit 20 and patient 12 may be, for example, a cath lab or an angio suite. Aseptic technique consists of using sterile barriers, sterile equipment, proper patient preparation, environmental controls and contact guidelines. Accordingly, all EMDs and interventional accessories are sterilized and can only be in contact with either sterile barriers or sterile equipment. In an embodiment, a sterile drape (not shown) is placed over the non-sterile robotic drive 24. Each cassette 66a-d is sterilized and acts as a sterile interface between the draped robotic drive 24 and at least one EMD. Each cassette 66a-d can be designed to be sterile for single use or to be re-sterilized in whole or part so that the cassette 66a-d or its components can be used in multiple procedures.

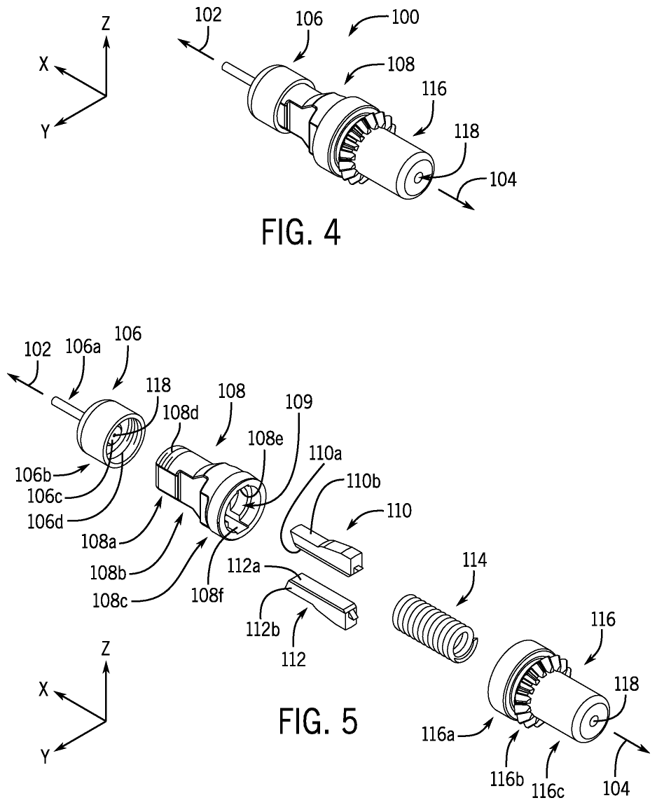

Referring to FIGS. 4 and 5 a passive torquer 100 in accordance with an embodiment includes an actuator 106, a body 108, a first jaw 110, a second jaw 112, a spring 114, and a spring housing 116. Torquer 100 includes a lumen 118 along the longitudinal centerline of torquer 100 extending throughout. Body 108 includes a cavity 109. Lumen 118 extends from a proximal end to a distal end of body 108 and is in fluid communication with cavity 109. Lumen 118 includes a lumen portion extending through knob 106 and a lumen portion extending through housing 116. The diameter of lumen 118 is sized to be larger than the diameter of the EMD (not shown in FIGS. 4 and 5) with which torquer 100 is used. As described herein, first jaw 110 and second jaw 112 are movable within cavity 109. In one embodiment spring 114 has a longitudinal axis that is co-linear with the longitudinal axis of body 108. In one embodiment actuator 106 is a knob movable relative to body 108 moves first jaw 110 to engage and/or disengage the EMD within cavity 109. Actuator 106 includes other known mechanisms and the term actuator and knob are used interchangeably herein. In one embodiment knob 106 movable relative to body 108 moves first jaw 110 and second jaw 112 to engage and/or disengage the EMD within cavity 109. Passive torquer 100 is normally in a closed position such that when an EMD is in torquer 100 it is pinched in the normally closed position. An operator or robotic system would need to act against the biasing member to unpinch the EMD.

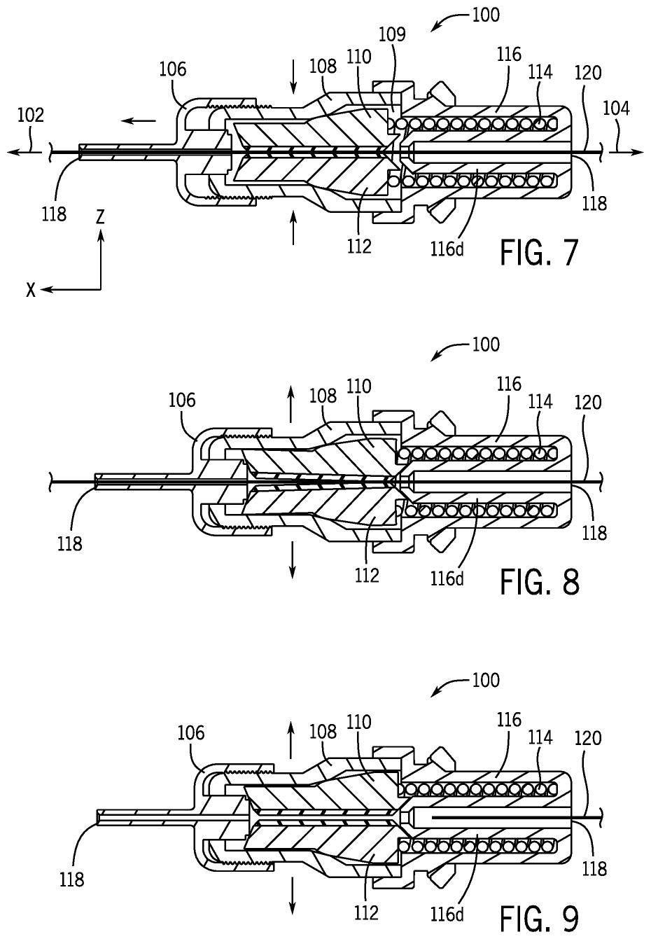

In the unpinched state of torquer 100, an EMD is inserted in lumen 118 at the distal end of torquer 100 in a longitudinal proximal direction 104 and is withdrawn or removed from lumen 118 at the distal end of torquer 100 in a longitudinal distal direction 102, or the EMD is withdrawn or removed from lumen 118 at the proximal end of torquer 100 in a longitudinal proximal direction. In one embodiment an EMD is inserted in lumen 118 at the proximal end of torquer 100 in a longitudinal distal direction 102 and is withdrawn from lumen 118 at either the proximal end of torquer 100 in a longitudinal proximal direction 104 or the distal end of torquer 100 in a longitudinal distal direction. In the pinched state of torquer 100 a portion of an EMD is fixed relative to torquer body 108. In particular, in the pinched state first jaw 110 and second jaw 112 of torquer 100 pinch a portion of the shaft of an EMD 120 (see FIG. 7) such that rotation and/or translation of torquer 100 about or along its longitudinal axis results in a distributed torque and or force along the pad imparting the same or substantially the same rotation and/or translation of the portion of the shaft of the EMD that is pinched. In one embodiment upon rotation of the torquer applying torque to the EMD when the EMD is pinched state the portion of the EMD along the longitudinal length of the pads there is progressively increasing torsion in the EMD from the proximal end of the torquer to the distal end of the torquer. In one embodiment the EMD is fixed relative to the proximal end of the pads in the pinched position. The EMD has a degree of rotation along the length of the pads from the proximal end to the distal end of the torquer when a torque is applied.

Knob 106 includes a distal portion 106a and a proximal portion 106b, with the longitudinal centerlines of both portions aligned with the longitudinal centerline of torquer 100. In one embodiment distal portion 106a of knob 106 is a support tube with a lumen 118 that extends distally to limit buckling and prevent kinking of a portion of the EMD along its length as the EMD is being translated and/or rotated. In one embodiment distal portion 106a of knob 106 is a cylindrical support tube with a lumen 118. In one embodiment proximal portion 106b of knob 106 is a cylindrical cup that is open in the proximal direction and has an interior protrusion 106c extending in the proximal direction from the distal base of the cylindrical cup. In one embodiment interior protrusion 106c of knob 106 is a cylinder with a lumen 118 and with its centerline aligned with the longitudinal centerline of torquer 100. In one embodiment proximal portion 106b of knob 106 includes internal screw threads 106d on the inner wall of a cylindrical cup. In one embodiment proximal portion 106b of knob 106 includes external screw threads on the outer wall of the interior protrusion 106c. In one embodiment 106 includes internal screw threads while 108d has external screw threads. In one embodiment knob 106 is a single manufactured component, such as a molded component, with lumen 118 as an internal pathway through which passes a portion of the shaft of the EMD. In one embodiment knob 106 is an assembled component, with lumen 118 as an internal pathway through which passes a portion of the shaft of the EMD. In one embodiment the pathway can accommodate elongated medical devices having a diameter of 0.014 inch (0.356 mm) through and including 0.038 inch (0.965 mm). In one embodiment the range of the diameters of the elongated medical devices that can be accommodated includes devices having diameters of 0.038 inch (0.965 mm) or less. In one embodiment the pathway can accommodate a specific diameter EMD with a range such as 0.016 inches (0.406 mm)+−0.002 inches (0.051 mm)

Body 108 includes a distal portion 108a, an intermediate portion 108b, and a proximal portion 108c, with the longitudinal centerlines of all portions aligned with the longitudinal centerline of torquer 100. In one embodiment body 108 is a hollow cylinder with distinct inner and outer diameters in the distal portion 108a, intermediate portion 108b, and proximal portion 108c. In one embodiment body 108 may have non-cylindrical features. In one embodiment the outer wall of distal portion 108a includes external screw threads 108d. In one embodiment the inner wall of distal portion 108a includes internal screw threads. In one embodiment the interior wall of body 108 includes a first channel 108e and a second channel 108f that are grooved cutouts in the interior wall located across from one another. The width of first channel 108e is larger than the width of first jaw 110 and the width of second channel 108f is larger than the width of second jaw 112. In one embodiment body 108 is a single manufactured component, such as a molded component, with an internal pathway through which passes a portion of the shaft of the EMD. In one embodiment body 108 is an assembled component, with an internal pathway through which passes a portion of the shaft of the EMD.

First jaw 110 includes a first pad 110a and a first pad base 110b and second jaw 112 includes a second pad 112a and a second pad base 112b. In one embodiment first pad base 110b is a parallelepiped like member with a longitudinal axis corresponding to its longest dimension oriented along the longitudinal axis of torquer 100. In one embodiment first pad base 110b is a cuboid-like member with a longitudinal axis corresponding to its longest dimension oriented along the longitudinal axis of torquer 100. In one embodiment first pad base has a prism-like shape. In one embodiment first pad base 110b includes a flat bottom surface to which first pad 110a is affixed. In one example pad 110a is chemically bonded to pad base 110b, mechanically attached or other known means of attaching members together. In one embodiment second pad base 112b is a parallelepiped-like member with a longitudinal axis corresponding to its longest dimension oriented along the longitudinal axis of torquer 100. In one embodiment second pad base 112b is a cuboid-like member with a longitudinal axis corresponding to its longest dimension oriented along the longitudinal axis of torquer 100. In one embodiment second pad base 112b includes a flat top surface to which second pad 112a is affixed.

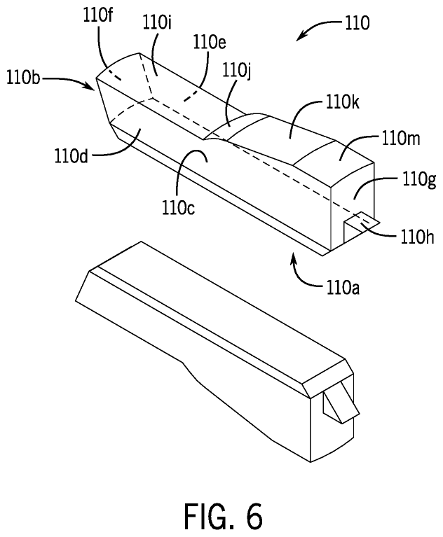

Referring to FIG. 6 one embodiment of first pad base 110b includes a flat bottom (lower) surface 110c to which first pad 110a is affixed, a flat front lateral surface 110d, a flat back lateral surface 110e, an inclined flat distal surface 110f, a flat proximal surface 110g with a protrusion 110h, and a top (upper) surface comprising a flat distal portion 110i, a curved intermediate portion 110j, an inclined flat intermediate portion 110k, and a flat proximal portion 110m. In one embodiment curved intermediate portion 110j of the top surface of first pad base 110b has a convex arcuate profile and is a transition surface between flat distal portion 110i and inclined flat intermediate portion 110k.

In one embodiment second pad base 112b is identical to first pad base 110b and includes surfaces respectively congruent to those of the surfaces of first pad base 110b. In one embodiment of torquer 100 second pad base 112b is rotated (flipped) 180 degrees about its longitudinal axis relative to first pad base 110b. In other words, flat bottom surface 110c of first pad base 110b to which first pad 110a is attached as described herein faces flat top surface of second pad base 112b to which second pad 112a is attached.

In one embodiment the bottom surface of first pad 110a of first jaw 110 is a flat surface. In one embodiment the bottom surface of first pad 110a of first jaw 110 is a flat surface that includes a concave arcuate profile (in a transverse plane, that is, in the Y-Z plane) extending along the length of first pad 110a surface. In one embodiment the bottom surface of first pad 110a of first jaw 110 is a curved surface with a concave arcuate profile (in a transverse plane, that is, in the Y-Z plane) extending along the length of first pad 110a surface.

In one embodiment second pad 112a is identical to first pad 110a and includes surfaces respectively congruent to those of the surfaces of first pad 110a. In one embodiment the top surface of second pad 112a of second jaw 112 is identical to the bottom surface of first pad 110a of first jaw 110 and includes surfaces respectively congruent to those of the surfaces of first pad 110a. In one embodiment first pad 110a is secured to first pad base 110b and second pad 112a is secured to second pad base 112b.

In one embodiment first pad 110a and second pad 112a are made of a medical-grade biocompatible material that does not damage or penetrate the coating on an EMD, such as a guidewire, used in a catheter procedure when pressed into it. In one embodiment first pad 110a and second pad 112a are made of an elastomeric material within a range of durometer measures of 50D-75D and manufactured with specific smoothness/roughness/texture ratings, such as SPI B1, A1, C1, A2, B2, or C2. In one embodiment each of the SPI (Society of Plastic Industry) ratings identified herein correspond with the following Ra (roughness parameter) values in microinches (μin) shown in parentheses after the identified SPI ratings: SPI B1 (RA 2-3), A1 (RA 0-1), A2 (RA 1-2), B2 (RA 4-5) and C2 (RA 25-28). In one embodiment first pad 110a and second pad 112a are made of a natural or synthetic material with low elastic modulus values and high strain values compared with other materials such as metallic materials.

An elastomeric material as used here is a material made of a polymer with elastic or viscoelastic properties, or a rubber or rubber-like material with elastic or viscoelastic properties, or a material that has compliant properties and/or elastic or viscoelastic properties. First pad 110a and second pad 112a are referred to herein as an elastomeric pad. In one embodiment each pad with a compliant property is formed from a polyurethane material or polyether block amide (PEBA) material.

In one embodiment first pad base 110b and second pad base 112b are made of a medical-grade biocompatible material, such as a biocompatible plastic, that is harder than the material of first pad 110a and second pad 112a. In one embodiment first pad base 110b and second pad base 112b are made of a material such as Ultem 1000 or stainless steel. In one embodiment first pad base 110b and second pad base 112b are made of a material that is more rigid than the material of the first pad 110a and second pad 112a. In one embodiment first pad base 110b and second pad base 112b are made of a material with an elastic modulus equal to or greater than a value of 3.5 GPa. In one embodiment first pad base 110b and second pad base 112b are made of a material with an elastic modulus with a value two or more times that of the material of first pad 110a and second pad 112a. In one embodiment first pad base 110b and second pad base 112b are made of a material with an elastic modulus with a value that is ten times or more that of the material of first pad 110a and second pad 112a.

In one embodiment of torquer 100 internal screw threads 106d of knob 106 mesh with external screw threads 108d of body 108 such that rotation of knob 106 relative to body 108 results in a change in the longitudinal distance between knob 106 and body 108, with the distance increasing or decreasing depending on the direction of relative rotation. The change in longitudinal distance per unit of relative rotation of knob 106 and body 108 is related to the pitch of the meshing threads 106d and 108d. In one embodiment (not shown) of torquer 100 external screw threads of interior protrusion 106c of knob 106 mesh with internal screw threads on the interior wall of the distal portion 108a of body 108 such that rotation of knob 106 relative to body 108 results in a change in the longitudinal distance between knob 106 and body 108, with the distance increasing or decreasing depending on the direction of relative rotation. The change in longitudinal distance per unit of relative rotation of knob 106 and body 108 is related to the pitch of the meshing threads.

In one embodiment spring 114 is a helical compression spring. In one embodiment spring 114 is a helical compression spring with plain and ground ends. In one embodiment spring 114 is a helical compression spring with squared and ground ends. In one embodiment spring 114 is a compliant elastic member in the shape of a hollow cylinder or another geometry.

Spring housing 116 includes a distal portion 116a, a bevel gear 116b, and a proximal portion 116c, with the longitudinal centerlines of all portions aligned with the longitudinal centerline of torquer 200. In one embodiment bevel gear 116b is intermediate the distal portion 116a and proximal portion 116c of spring housing 116 and integrally affixed to portions 116a and 116c. In one embodiment the teeth of bevel gear 116b are oriented in the longitudinal proximal direction 104. In one embodiment bevel gear is a driven member operatively driven by a drive member in a robotic system. In one embodiment distal portion 116a of spring housing 116 is a cylindrical cup that is open in the distal direction and includes an opening in its proximal base. In one embodiment proximal portion 116c of spring housing 116 is a cylindrical cup that is open in the distal direction and has an interior post 116d extending in the distal direction from the proximal base of the cylindrical cup with a lumen 118 extending throughout that is aligned with the longitudinal central axis of torquer 100. In one embodiment the interior post 116d of spring housing 116 is a cylindrical protrusion with a chamfered distal end, with a central lumen 118, and with its centerline aligned with the longitudinal centerline of torquer 100. In one embodiment spring housing 116 is a single manufactured component, such as a molded component, with lumen 118 as an internal pathway through which passes a portion of the shaft of the EMD. In one embodiment spring housing 116 is an assembled component, with lumen 118 as an internal pathway through which passes a portion of the shaft of the EMD. In one embodiment bevel gear 116b may be a driven member that can be located on any outer portion of the torquer body and/or may be located on an outer portion of the actuator or knob 106. Driven member 116b may be another type of gear such as a spur gear, worm gear, hypoid gear or could be a surface that frictionally engages a drive member including but not limited to a belt drive mechanism.

In one embodiment of assembled torquer 100 the proximal portion 108c of body 108 is snap fit to the distal portion 116a of housing 116, for example, via a molded undercut on one part that engages with a mating lip on the other. In one embodiment of assembled torquer 100 the proximal portion 108c of body 108 is press fit to the distal portion 116a of housing 116, for example, using dimensional interferences on the mating parts. In one embodiment the proximal portion 108c of body 108 is fixed to the distal portion 116a of housing 116 by glue, adhesive, bonding agent, laser welding, ultrasonic welding, or other means of affixing two bodies during assembly and manufacture. In one embodiment of torquer 100 body 108 is removably fixed to housing 116 using fasteners (not shown). The term snap fit as used herein is an assembly method used to attach flexible parts, usually plastic, to form the final product by pushing the parts' interlocking components together. There are a number of variations in snap fits, including cantilever, torsional and annular. Snap fits, as integral attachment features, are an alternative to assembly using nails or screws, and have the advantages of speed and no loose parts.

In one embodiment of torquer 100 spring 114 is constrained from lateral or transverse motion (that is, motion in a Y-Z plane) relative to housing 116 and prevented from buckling by being seated over a central post 116d extending in the distal direction from the proximal base of the cylindrical cup of housing 116, where the central post 116d is of cylindrical shape with an outer diameter less than the inner diameter of spring 114 and includes a central lumen 118. In one embodiment the inner diameter of the proximal portion 116c of housing 116 or the outer diameter of the inner post 116d of the proximal portion 116c of housing 116 are needed to prevent buckling.

In one embodiment of torquer 100 the proximal end of spring 114 is constrained from longitudinal motion relative to housing 116 by contact with the interior surface of the cylindrical cup base at the proximal end of the proximal portion 116c of housing 116. In one embodiment of torquer 100 the distal end of spring 114 is in contact with the flat proximal surface 110g of first pad base 110b of first jaw 110 and in contact with the flat proximal surface of second pad base 112b of second jaw 112. In one embodiment first pad base 110b includes a wedge protrusion 110h on the flat proximal surface 110g close to the bottom surface 110c of first pad base 110b and second pad base 112b includes a corresponding wedge protrusion on the flat proximal surface close to the top surface of second pad base 112b, with both wedge protrusions extending proximally and with both wedge protrusions located within the inner diameter of spring 114 at its distal end.

In one embodiment of torquer 100 first pad base 110b is kinematically constrained in first channel 108e of body 108 and second pad base 112b is kinematically constrained in second channel 108f of body 108. In particular, in one embodiment the walls of first channel 108e constrain lateral motion of first jaw 110 (by contacting flat front lateral surface 110d and flat back lateral surface 110e of first pad base 110b) and the walls of second channel 108f constrain lateral motion of second jaw 112.

In one embodiment a portion of the top surface of first pad base 110b of first jaw 110 contacts a portion of the inner circumferential wall of first channel 108e of body 108 and a portion of the bottom surface of second pad base 112b of second jaw 112 contacts a portion of the inner circumferential wall of second channel 108f of body 108.

In one embodiment torquer 100 includes two jaws that move relative to each other to releasably fix a portion of the shaft of the EMD to at least one of the two jaws. In one embodiment torquer 100 includes one jaw that moves relative to the body of the torquer 100 to releasably pinch a portion of the shaft of the EMD to the one jaw. In one embodiment torquer 100 includes more than two jaws that move relative to each other to releasably fix a portion of the shaft of the EMD to at least one of the jaws.

In one embodiment of torquer 100 spring 114 acts as a biasing member that biases one jaw relative to the body. In one embodiment of torquer 100 spring 114 acts as a biasing member that biases two jaws relative to the body. In one embodiment of torquer 100 spring 114 acts as a biasing member that biases more than two jaws relative to the body.

In one embodiment two or more members operating together provide a mechanical advantage that increases the torque and/or force that may be transmitted from the torquer to a portion of the shaft of the EMD without the shaft of the EMD moving relative to the torquer. The pinch force on the EMD using a torquer can be greater than the force required to actuate the pinch. When a portion of the shaft of the EMD is pinched it is fixed such that there is no relative movement of the torquer and that portion of the EMD during acceptable operation parameters of an EMD procedure.

Referring to FIGS. 7, 8 and 9 passive torquer 100 in accordance with an embodiment is shown in stages corresponding to a pinched state, a partially pinched state, and an unpinched state, respectively. In a pinched state torquer 100 is in a fully engaged position and is pinching a portion of an EMD 120, in a partially pinched state torquer 100 is in a partially engaged position and is partially pinching a portion of an EMD 120, and in an unpinched state torquer 100 is in a disengaged position and is not pinching an EMD 120. In the embodiment depicted in all three states (pinched, partially pinched, and unpinched) the internal threads 106d of knob 106 mesh with the external threads 108d of body 108. In the unpinched state the distance between the pads in a direction perpendicular to the longitudinal axis of the torquer is greater than the diameter of the EMD.

Referring to FIG. 7 in the pinched state of torquer 100 knob 106 is in an open position relative to body 108. There is no contact (that is, a gap exists) between the proximal surface of the interior protrusion 106c of knob 106 and the inclined distal surface 110f of first pad base 110b of first jaw 110 and there is no contact between the proximal surface of the interior protrusion 106c of knob 106 and the inclined distal surface of second pad base 112b of second jaw 112. Rotating knob 106 relative to body 108 in a direction that unthreads knob 106 from body 108 causes knob 106 to move in a longitudinal distal direction 102 relative to body 108 increasing the gap between the proximal surface of interior protrusion 106c and the distal surfaces of first jaw 110 and of second jaw 112.

In one embodiment knob 106 freely rotates relative to body 108 in a direction that unthreads knob 106 from body 108 until their teeth no longer mesh and knob 106 separates from body 108. In one embodiment knob 106 freely rotates relative to body 108 in a direction that unthreads knob 106 from body 108 until a stop is reached preventing knob 106 from separating from body 108.

In the pinched state of torquer 100 in which knob 106 is in an open position relative to body 108 there is also no contact (that is, a gap exists) between the inclined surface of wedge protrusion 110h on the proximal end of first pad base 110b and the distal chamfered surface that faces it on the central post 116d (extending in the distal direction from the proximal base of the cylindrical cup) of spring housing 116 and there is no contact between the inclined surface of wedge protrusion on the proximal end of second pad base 112b and the distal chamfered surface that faces it on the central post 116d of spring housing 116.

In the pinched state of torquer 100 first pad 110a of first jaw 110 and second pad 112a of second jaw 112 face each other, are parallel to each other and to a portion of EMD 120, and pinch a portion of EMD 120 over the length of each pad, that is, first pad 110a and second pad 112a are in contact with a portion of EMD 120 over the length of each pad.

In the pinched state of torquer 100 spring 114 is compressed relative to its rest length. As a result, a spring restoring force acts in the longitudinal distal direction 102. (A spring restoring force also acts in the longitudinal proximal direction 104 for static equilibrium. However, the proximal end of spring 114 is constrained, that is, fixed relative to housing 116 and to body 108 to which housing 116 is affixed. Thus, the spring restoring force of interest acts in the longitudinal distal direction 102.) Half of this force acts on first jaw 110 by contact between the distal end of spring 114 and the flat proximal surface 110g of first pad base 110b, and half of this force acts on second jaw 112 by contact between the distal end of spring 114 and the flat proximal surface of second pad base 112b.

Although a force (half of the restoring force from spring 114) is applied in the longitudinal distal direction 102 to first jaw 110, first jaw 110 is constrained from moving relative to body 108 in the longitudinal distal direction 102. Motion of first jaw 110 in the longitudinal distal direction 102 is constrained by a force component of equal magnitude and opposite direction to that of half of the restoring force from spring 114. That is, a force component acts in the longitudinal proximal direction 104 to achieve static equilibrium of first jaw 110 along a longitudinal direction. This longitudinal force component acts at the contact point or region between the curved intermediate portion 110j of the top surface of first pad base 110b of first jaw 110 and the profiled portion of the top inside surface of first channel 108e of body 108.

A vertical force component also acts at the contact point or region between the curved intermediate portion 110j of the top surface of first pad base 110b of first jaw 110 and the profiled portion of the top inside surface of first channel 108e of body 108, as described herein.

The profiled portion of the top inside surface of first channel 108e of body 108 defines a cam surface that contacts the curved intermediate portion 110j of the top surface of first pad base 110b of first jaw 110 that defines a follower surface. Due to the profiling of the cam-follower surfaces (and the force from the spring), a resultant force acts on first jaw 110 from body 108 at the contact point or region with a longitudinal force component directed proximally (in the negative X direction) and a vertical force component directed down (in the negative Z direction). As a result of the vertical force component acting on first jaw 110, first pad 110a is pressed into a portion of EMD 120 and there is contact between a portion of EMD 120 and first pad 110a. In one embodiment the follower surface is nonlinear. In one embodiment the follower surface is linear. In one embodiment the follower surface is arcuate. In one embodiment body 108 includes a cam surface contacting a nonlinear follower surface on first pad base 110b. In one embodiment body 108 includes a cam surface contacting a linear follower surface on first pad base 110b. In one embodiment body 108 includes a cam surface contacting an arcuate follower surface on first pad base 110b.

First jaw 110 can pivot and/or rock (in the X-Z plane) about the point or region of contact at the cam-follower surfaces. As contact develops between a portion of EMD 120 and first pad 110a, first jaw 110 pivots and/or rocks about the point or region of contact distributing the vertical force component acting on a portion of EMD 120 and equalizing the pressure on EMD 120 along the entire length of first pad 110a. The term pivot and/or rock includes both moving about a single point as well as moving about a surface along a predefined profile.

Similarly, a force (half of the restoring force from spring 114) is applied in the longitudinal distal direction 102 to second jaw 112, which is constrained from moving relative to torquer body 108 in the longitudinal distal direction 102. Motion of second jaw 112 in the longitudinal distal direction 102 is constrained by a force component of equal magnitude and opposite direction to that of half of the restoring force from spring 114. That is, a force component acts in the longitudinal proximal direction 104 to achieve static equilibrium of second jaw 112 along a longitudinal direction. This longitudinal force component acts at the contact point or region between the curved intermediate portion of the bottom surface of second pad base 112b of second jaw 112 and the profiled portion of the bottom inside surface of second channel 108f of body 108.

A vertical force component also acts at the contact point or region. The profiled portion of the bottom inside surface of second channel 108f of body 108 defines a cam surface that contacts the curved intermediate portion of the bottom surface of second pad base 112b of second jaw 112 that defines a follower surface. Due to the profiling of the cam-follower surfaces (and the force from the spring), a resultant force acts on second jaw 112 from body 108 at the contact point or region with a longitudinal force component directed proximally (in the negative X direction) and a vertical force component directed up (in the positive Z direction). As a result of the vertical force component acting on second jaw 112, second pad 112a is pressed into a portion of EMD 120 and there is contact between a portion of EMD 120 and second pad 112a. In one embodiment the follower surface is nonlinear. In one embodiment the follower surface is linear. In one embodiment the follower surface is arcuate. In one embodiment body 108 includes a cam surface contacting a nonlinear follower surface on second pad base 112b. In one embodiment body 108 includes a cam surface contacting a linear follower surface on second pad base 112b. In one embodiment body 108 includes a cam surface contacting an arcuate follower surface on second pad base 112b.

Second jaw 112 can pivot and/or rock (in the X-Z plane) about the point or region of contact at the cam-follower surfaces. As contact develops between a portion of EMD 120 and second pad 112a, second jaw 112 pivots and/or rocks about the point or region of contact distributing the vertical force component acting on a portion of EMD 120 and equalizing the pressure on EMD 120 along the length of second pad 112a.

First jaw 110 and second jaw 112 are free to pivot and/or rock about a cam surface on body 108 independent of one another and wherein first jaw 110 and second jaw 112 are not connected to one another. In one embodiment first jaw 110 and second jaw 112 are not directly connected to each other as a single manufactured component. In one embodiment first jaw 110 and second jaw 112 are not directly connected to each other via a linkage. In one embodiment a distal end and a proximal end of first pad 110a moves radially away from the longitudinal axis of torquer 100 as first jaw 110 pivots about the cam surface. In one embodiment a distal end and a proximal end of first pad 110a and a distal end and a proximal end of second pad 112a move radially away from the longitudinal axis of torquer 100 as first jaw 110 and second jaw 112 pivot about the cam surfaces.

As first pad 110a and second pad 112a are pressed toward one another, and each into EMD 120 with a circular cross-section, first pad 110a and second pad 112a each deform slightly around EMD 120 and there is contact between the bottom surface of first pad 110a and a portion of the circumference of EMD 120 over the length of first pad 110a and there is contact between the top surface of second pad 112a and a portion of the circumference of EMD 120 over the length of second pad 112a. In one embodiment pressure is equalized on EMD 120 along the length of the elastomeric pads contacting the portion of EMD 120 in a fully engaged position. In one embodiment pressure is equalized on EMD 120 along the entire length of the elastomeric pads contacting the portion of EMD 120 in a fully engaged position. In one embodiment pressure is equalized on EMD 120 along the majority of the length of the elastomeric pads contacting the portion of EMD 120 in a fully engaged position. In one embodiment pressure between the elastomeric pads and a portion of EMD 120 is substantially equalized along an entire length of the elastomeric pads in a fully engaged position.

As the first pad 110a deforms partially around EMD 120 and second pad 112a deforms partially around EMD 120, that is, each conforming to an arc of the circular cross-section of EMD 120 over their lengths, they are pressed toward one another from opposite directions and EMD 120 is pinched between them.

Referring to FIG. 8 in the partially pinched state of torquer 100 knob 106 is in a “partially closed” position relative to body 108. Torquer 100 partially pinches EMD 120 in the transition from the pinched state to the unpinched state. Rotating knob 106 relative to body 108 in a direction that threads knob 106 toward body 108 causes knob 106 to move in a longitudinal proximal direction 104 relative to body 108 such that no gap exists between the proximal surface of interior protrusion 106c and the distal surfaces of first jaw 110 and of second jaw 112. In particular, there is contact (that is, no gap exists) between the proximal surface of the interior protrusion 106c of knob 106 and the inclined distal surface 110f of first pad base 110b of first jaw 110 and there is contact between the proximal surface of the interior protrusion 106c of knob 106 and the inclined distal surface of second pad base 112b of second jaw 112.

As knob 106 is threaded toward body 108 the proximal surface of the interior protrusion 106c of knob 106 moves in the longitudinal proximal direction 104 and it pushes in the longitudinal proximal direction 104 against the inclined distal surface 110f of first pad base 110b of first jaw 110. As the proximal surface of the interior protrusion 106c pushes against the inclined distal surface 110f of first jaw 110 in the longitudinal proximal direction 104, due to the orientation (slant angle) of the inclined distal surface 110f of first jaw 110 the distal end of first pad 110a of first jaw 110 moves radially away from EMD 120 and separates from it. Similarly, as the proximal surface of the interior protrusion 106c pushes against the inclined distal surface of second jaw 112 in the longitudinal proximal direction 104, due to the orientation (slant angle) of the inclined distal surface of second jaw 112 the distal end of second pad 112a of second jaw 112 moves radially away from EMD 120 and separates from it.

In the partially pinched state, as knob 106 is threaded toward body 108 the proximal surface of the interior protrusion 106c of knob 106 moves in the longitudinal proximal direction 104 pushing on and moving first jaw 110 and second jaw 112 proximally relative to the positive of first jaw 110 and second jaw 112 in the pinched state. This compresses spring 114 further than it is compressed in the pinched state giving rise to a spring restoring force in the longitudinal distal direction 102 that is of larger magnitude than that in the pinched state.

In the partially pinched state of torquer 100 in which knob 106 is in a partially closed position relative to body 108 there is also contact (that is, no gap exists) between the inclined surface of wedge protrusion 110h on the proximal end of first pad base 110b and the distal chamfered surface facing it on the central post 116d (extending in the distal direction from the proximal base of the cylindrical cup) of spring housing 116 and there is contact between the inclined surface of wedge protrusion on the proximal end of second pad base 112b and the distal chamfered surface facing it on the central post 116d of spring housing 116.

In the partially pinched state of torquer 100 first pad 110a of first jaw 110 and second pad 112a of second jaw 112 are in a non-aligned position relative to a longitudinal central axis of torquer 100. In the partially pinched state of torquer 100 first pad 110a of first jaw 110 and second pad 112a of second jaw 112 are not parallel and are partially pinching and partially unpinching portions of EMD 120. In particular, due to their non-aligned positions first pad 110a and second pad 112a are in contact (or partial contact) toward their proximal ends with a portion of EMD 120 and first pad 110a and second pad 112a are not in contact toward their distal ends with a portion of EMD 120. In one embodiment one of the distal ends and proximal ends of the jaws move away from one another prior to the other of the distal ends and proximal ends of the jaws.

As described above, the profiled portion of the top inside surface of first channel 108e of body 108 defines a cam surface and the curved intermediate portion 110j of the top surface of first pad base 110b of first jaw 110 defines a follower surface. A longitudinal force component acts on first pad base 110b of first jaw 110 in the longitudinal proximal direction 104 to achieve static equilibrium in the longitudinal direction with half of the restoring from spring 114. This longitudinal force component is of larger magnitude than that which develops in the pinched state. Due to the profiling of the cam-follower surfaces, a vertical force component also acts on first pad base 100b of first jaw 110. The longitudinal force component is directed proximally and the vertical force component is directed down. In the partially pinched state of torquer 100 the vertical force component presses a proximal portion of first pad 110a into EMD 120 and there is contact between a portion of EMD 120 and a proximal portion of first pad 110a.

Similarly, the profiled portion of the bottom inside surface of second channel 108f of body 108 defines a cam surface and the curved intermediate portion of the bottom surface of second pad base 112b of second jaw 112 defines a follower surface.

A longitudinal force component acts on second pad base 112b of second jaw 112 in the longitudinal proximal direction 104 to achieve static equilibrium in the longitudinal direction with half of the restoring from spring 114. This longitudinal force component is of larger magnitude than that which develops in the pinched state. Due to the profiling of the cam-follower surfaces, a vertical force component also acts on second pad base 112b of second jaw 112. The longitudinal force component is directed proximally and the vertical force component is directed up. In the partially pinched state of torquer 100 the vertical force component presses a proximal portion of second pad 112a into EMD 120 and there is contact between a portion of EMD 120 and a proximal portion of second pad 112a.

A proximal portion of first pad 110a and a proximal portion of second pad 112a each deforms around a portion of EMD 120. The proximal portions of first pad 110a and second pad 112a are pressed toward one another from opposite directions partially pinching a portion of EMD 120 between them.

Referring to FIG. 9 in the unpinched state of torquer 100 knob 106 is in a closed position relative to body 108. In the unpinched state of torquer 100 EMD 120 in lumen 118 can be withdrawn in a longitudinal proximal direction 104 or EMD 120 can be inserted in lumen 118 in a longitudinal distal direction 102. Fully rotating knob 106 relative to body 108 in a direction that threads knob 106 toward body 108 until no further travel is possible causes knob 106 to move to its most proximal position relative to body 108. As with the partially pinched state no gap exists between the proximal surface of interior protrusion 106c and the distal surfaces of first jaw 110 and of second jaw 112.

In the fully unpinched state of torquer 100 the proximal surface of the interior protrusion 106c of knob 106 is in its most proximal position. The proximal surface (or an edge of the proximal surface) of the interior protrusion 106c of knob 106 contacts the inclined distal surface 110f of first jaw 110 at a portion of the inclined distal surface 110f of first jaw 110 at its most radially distant position from the central axis. Due to the orientation (slant angle) of the inclined distal surface 110f of first jaw 110 the distal end of first pad 110a of first jaw 110 moves to its most radially distant position away from the central longitudinal axis of torquer 100. Similarly, the proximal surface (or an edge of the proximal surface) of the interior protrusion 106c of knob 106 contacts the inclined distal surface of second jaw 112 at a portion of the inclined distal surface of second jaw 112 at its most radially distant position from the central axis. Due to the orientation (slant angle) of the inclined distal surface of second jaw 112 the distal end of second pad 112a of second jaw 112 moves to its most radially distant position away from the central longitudinal axis of torquer 100.

In the fully unpinched state of torquer 100 the proximal surface (or an edge of the proximal surface) of the interior protrusion 106c of knob 106 pushes first jaw 110 and second jaw 112 in the longitudinal proximal direction 104 to their most proximal achievable positions, corresponding to maximum compression of spring 114 of torquer 100. A maximum restoring force from spring 114 of torquer 100 develops and acts in the longitudinal distal direction 102 on first jaw 110 and second jaw 112.

In the fully unpinched state of torquer 100 the inclined surface of wedge protrusion 110h on the proximal end of first pad base 110b is in its most proximal position and is pushing against on the distal chamfered surface in contact with it on the central post 116d (extending in the distal direction from the proximal base of the cylindrical cup) of spring housing 116. Due to the orientation (slant angle) of the inclined surface of wedge protrusion 110h on the proximal end of first pad base 110b the proximal end of first pad 110a of first jaw 110 moves radially away from the central longitudinal axis of torquer 100. Similarly, the inclined surface of the wedge protrusion on the proximal end of second pad base 112b is in its most proximal position and is pushing against on the distal chamfered surface in contact with it on the central post 116d of spring housing 116. Due to the orientation (slant angle) of the inclined surface of the wedge protrusion on the proximal end of second pad base 112b the proximal end of second pad 112a of second jaw 112 moves radially away from the central longitudinal axis of torquer 100.

In the fully unpinched state of torquer 100 both the proximal end and distal end of first jaw 110 and of second jaw 112 are in positions most radially distant from the central longitudinal axis of torquer 100, creating a gap distance between the opposing outermost surfaces of first pad 110a and second pad 112a along their lengths that is larger than the diameter of EMD 120. In other words, first pad 110a of first jaw 110 and second pad 112a of second jaw 112 are in a disengaged position and torquer 100 is not pinching EMD 120.

In one embodiment of torquer 100 the 0.014 inch (0.356 mm) guidewire torque target is greater than or equal to 2 mNm. Stated another way in one embodiment torquer 100 transmits or applies greater than or equal to 2 mNm without the EMD slipping relative to the torquer. In one embodiment of torquer 100 the elastomeric pad length of each pad is less than 50 mm. In one embodiment of torquer 100 the elastomeric pad thickness is in the range of 0.5 mm to 2 mm. In one embodiment of torquer 100 the elastomeric pad modulus is in the range of 250 MPa to 320 MPa. In one embodiment of torquer 100 the pad base material is a stainless steel. In one embodiment of torquer 100 the pad bad modulus is greater than or equal to 3 GPa.

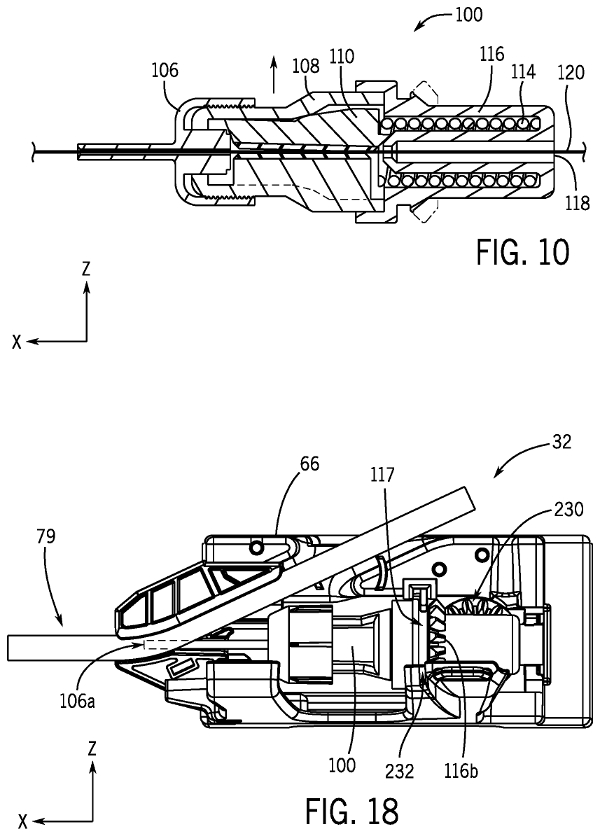

Referring to FIG. 10 an embodiment of passive torquer 100 includes a movable first jaw 110 and a fixed second jaw 112. In one embodiment passive torquer 100 includes more than two movable jaws. In one embodiment passive torquer 100 does not include bevel gear 116b on housing 116, and housing 116 is manipulated manually. In one embodiment passive torquer 100 does not include bevel gear 116b on housing 116, and includes a mechanical means of rotation such as a pulley.

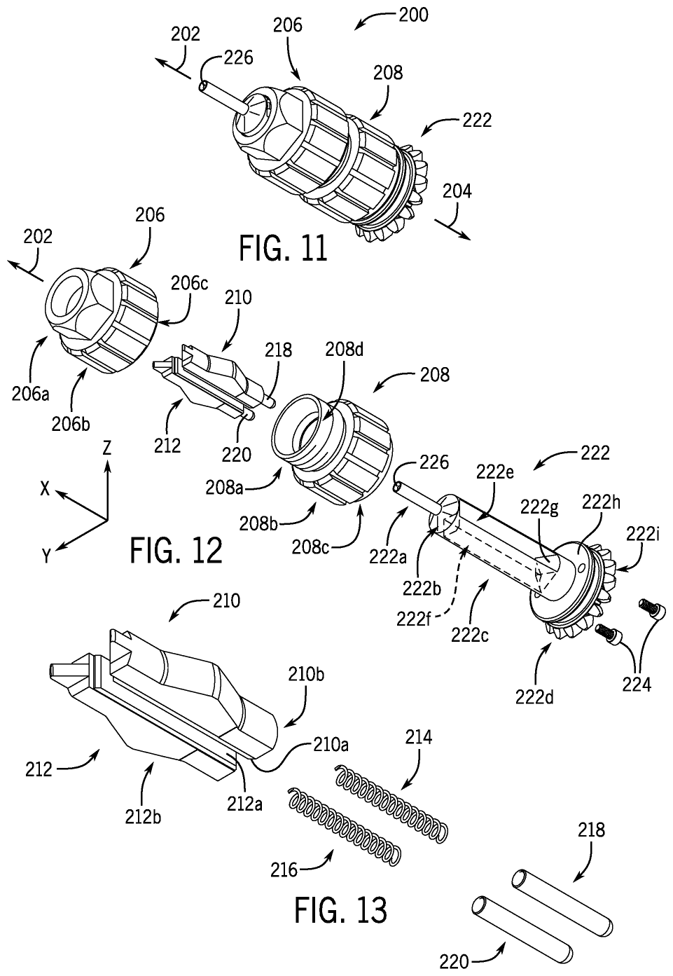

Referring to FIGS. 11, 12, and 13 an active torquer 200 in accordance with an embodiment includes a knob 206, a body 208, a first jaw 210, a second jaw 212, a first spring 214, a second spring 216, a first pin 218, a second pin 220, a housing 222, and fasteners 224. Torquer 200 includes a lumen 226 along the longitudinal centerline of torquer 200 extending throughout. The diameter of lumen 226 is sized to be larger than the diameter of the EMD with which torquer 200 is used.

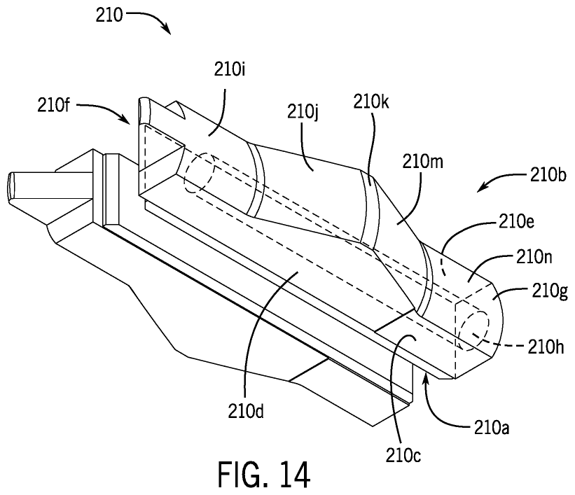

In the unpinched state of torquer 200, an EMD is inserted in lumen 226 at the distal end of torquer 200 in a longitudinal proximal direction 204 and is withdrawn from lumen 226 at the distal end of torquer 200 in a longitudinal distal direction 202, or an EMD is inserted in lumen 226 at the proximal end of torquer 200 in a longitudinal distal direction 202 and is withdrawn from lumen 226 at the proximal end of torquer 200 in a longitudinal proximal direction 204. As discussed above with respect to the passive torquer the EMD can be removed from the torquer from either the distal or proximal end independent of how the EMD was inserted into the torquer. In the pinched state of torquer 200 a portion of an EMD is fixed relative to torquer 200. In particular, in the pinched state first jaw 210 and second jaw 212 of torquer 200 pinch a portion of the shaft of an EMD 228 (see FIG. 14) such that rotation and/or translation of torquer 200 about or along its longitudinal axis results in the same rotation and/or translation of the portion of the shaft of the EMD that is pinched. Active torquer 200 is normally in the open or disengaged position such that an EMD is unpinched. An operator needs to move an actuator to over come the spring bias to close the torquer to pinch an EMD.

Knob 206 includes a distal portion 206a and a proximal portion 206b, with the longitudinal centerlines of both portions aligned with the longitudinal centerline of torquer 200. In one embodiment knob 206 is a hollow cylinder with distinct inner and outer diameters in the distal portion 206a and proximal portion 206b. In one embodiment distal portion 206a of knob 206 is a hollow cylinder. In one embodiment distal portion 206a of knob 206 is a hollow cylinder with an arrangement of flat outer faces on the outer wall, for example, in the shape of a hexagonal nut with its internal threads absent. In one embodiment distal portion 206a of knob 206 is a hollow cylinder with an arrangement of profiled faces on the outer wall. In one embodiment distal portion 206a of knob 206 is a hollow cylinder with a smooth inner wall. In one embodiment proximal portion 206b of knob 206 is a hollow cylinder with internal screw threads 206c, that is screw threads on the inner wall of the cylinder. In one embodiment proximal portion 206b of knob 206 is a hollow cylinder with an arrangement of profiled faces on the outer wall. In one embodiment proximal portion 206b of knob 206 is a hollow cylinder with a smooth outer wall. In one embodiment proximal portion 206b of knob 206 is a hollow cylinder with a knurled outer wall. In one embodiment knob 206 is a single manufactured component, such as a molded component with an internal pathway through which passes a portion of the shaft of an EMD. In one embodiment knob 206 is an assembled component with an internal pathway through which passes a portion of the shaft of an EMD.

Body 208 includes a distal portion 208a, an intermediate portion 208b, and a proximal portion 208c, with the longitudinal centerlines of all portions aligned with the longitudinal centerline of torquer 200. In one embodiment body 208 is an open cylinder with distinct diameters in the distal portion 208a, intermediate portion 208b, and proximal portion 208c. In one embodiment the outer wall of distal portion 208a includes external screw threads 208d. In one embodiment a portion of the inner wall of distal portion 208a includes a conical section, that is, a section of linearly increasing diameters or a section of linearly decreasing diameters in the plane transverse to a longitudinal axis (that is, Y-Z plane). In one embodiment the inner wall of distal portion 208a includes portions with conical sections, that is, sections of linearly increasing diameters and sections of linearly decreasing diameters in the plane transverse to a longitudinal axis (that is, Y-Z plane).

In one embodiment intermediate portion 208b of body 208 is a hollow cylinder with an arrangement of profiled faces on the outer wall. In one embodiment intermediate portion 208b of body 208 is a hollow cylinder with a smooth outer wall. In one embodiment intermediate portion 208b of body 208 is a hollow cylinder with a knurled outer wall. In one embodiment proximal portion 208c of body 208 includes threaded recessed holes on its most proximal face which receives fasteners 224. In one embodiment body 208 is a single manufactured component, such as a molded component, with an internal pathway through which passes a portion of the shaft of the EMD. In one embodiment body 208 is an assembled component, with an internal pathway through which passes a portion of the shaft of the EMD.

First jaw 210 includes a first pad 210a and a first pad base 210b and second jaw 212 includes a second pad 212a and a second pad base 212b. In one embodiment first pad 210a is secured to first pad base 210b and second pad 212a is secured to second pad base 212b. In one embodiment first pad base 210b is a parallelepiped-like member with a longitudinal axis corresponding to its longest dimension oriented along the longitudinal axis of torquer 200. In one embodiment first pad base 210b is a cuboid-like member with a longitudinal axis corresponding to its longest dimension oriented along the longitudinal axis of torquer 200. In one embodiment first pad base 210b includes a flat bottom surface to which first pad 210a is affixed. In one embodiment second pad base 212b is a parallelepiped-like member with a longitudinal axis corresponding to its longest dimension oriented along the longitudinal axis of torquer 200. In one embodiment second pad base 212b is a cuboid-like member with a longitudinal axis corresponding to its longest dimension oriented along the longitudinal axis of torquer 200. In one embodiment second pad base 212b includes a flat top surface to which second pad 212a is affixed.

Referring to FIG. 14 one embodiment of first pad base 210b includes a flat bottom (lower) surface 210c to which first pad 210a is affixed, a flat front lateral surface 210d, a flat back lateral surface 210e, an inclined distal surface 210f, a proximal surface 210g with a hole 210h extending distally within but not through to the distal surface of first pad base 210b, and a top (upper) surface comprising a distal portion 210i, a first intermediate portion 210j, a second intermediate portion 210k, a third intermediate portion 210m, and a proximal portion 210n. In one embodiment a transition portion is included between distal portion 210i and first intermediate portion 210j of the top surface of first pad base 210b and a transition portion is included between the third intermediate portion 210m and the proximal portion 210n of top surface of first pad base 210b. In one embodiment inclined distal surface 210f extends across the front distal face of first pad base 210b. In one embodiment inclined distal surface 210f comprises a portion of the front distal face of first pad base 210b. In one embodiment the diameter of hole 210h is larger in size than the outer diameter of first spring 214 and larger in size than the outer diameter of first pin 218. In one embodiment the top (upper) surface of portions 210i, 210j, 210k, 210m, and 210n of first pad base 210b are curved, such as circumferentially arcuate surfaces. In one embodiment the top (upper) surface of portions 210i, 210j, 210k, 210m, and 210n of first pad base 210b are flat surfaces.

In one embodiment second pad base 212b is identical to first pad base 210b and includes surfaces respectively congruent to those of the surfaces of first pad base 210b. In one embodiment of torquer 200 second pad base 212b is rotated (flipped) 180 degrees about its longitudinal axis relative to first pad base 210b. In other words, flat bottom surface 210c of first pad base 210b to which first pad 210a is attached faces flat top surface of second pad base 212b to which second pad 212a is attached.

In one embodiment the bottom surface of first pad 210a of first jaw 210 is a flat surface. In one embodiment the bottom surface of first pad 210a of first jaw 210 is a flat surface that includes a concave arcuate profile (in a transverse plane, that is, in the Y-Z plane) extending along the length of first pad 210a surface. In one embodiment the bottom surface of first pad 210a of first jaw 210 is a curved surface with a concave arcuate profile (in a transverse plane, that is, in the Y-Z plane) extending along the length of first pad 210a surface.

In one embodiment second pad 212a is identical to first pad 210a and includes surfaces respectively congruent to those of the surfaces of first pad 210a. In one embodiment the top surface of second pad 212a of second jaw 212 is identical to the bottom surface of first pad 210a of first jaw 210 and includes surfaces respectively congruent to those of the surfaces of first pad 210a. In one embodiment first pad 210a is secured to first pad base 210b and second pad 212a is secured to second pad base 212b.

In one embodiment first pad 210a and second pad 212a are made of a medical-grade biocompatible material that does not damage or penetrate the coating on an EMD, such as a guidewire, used in a catheter procedure when pressed into it. In one embodiment first pad 210a and second pad 212a are made of an elastomeric material within a range of durometer measures of 50D-75D and with specific smoothness/roughness ratings, such as SPI B1, A1, C1, A2, B2, or C2. In one embodiment first pad 210a and second pad 2112a are made of a natural or synthetic material with low elastic modulus values and high strain values compared with other materials.

In one embodiment first pad base 210b and second pad base 212b are made of a medical-grade biocompatible material, such as a biocompatible plastic, that is harder than the material of first pad 210a and second pad 212a. In one embodiment first pad base 210b and second pad base 212b are made of a material such as Ultem 1000 or stainless steel. In one embodiment first pad base 210b and second pad base 212b are made of a material that is more rigid than the material of the first pad 210a and second pad 212a. In one embodiment first pad base 210b and second pad base 212b are made of a material with an elastic modulus with a value equal to or greater than 3.5 GPa. In one embodiment first pad base 210b and second pad base 212b are made of a material with an elastic modulus with a value two or more times that of the material of first pad 210a and second pad 212a. In one embodiment first pad base 210b and second pad base 212b are made of a material with an elastic modulus with a value that is ten times that of the material of first pad 210a and second pad 212a.

In one embodiment of torquer 200 internal screw threads 206d of knob 206 mesh with external screw threads 208d of body 208 such that rotation of knob 206 relative to body 208 results in a change in the longitudinal distance between knob 206 and body 208, with the distance increasing or decreasing depending on the direction of relative rotation. The change in longitudinal distance per unit of relative rotation of knob 206 and body 208 is related to the pitch of the meshing threads 206d and 208d. In one embodiment of torquer 200 external screw threads of knob 106 mesh with internal screw threads on the interior wall of the distal portion 208a of body 208 such that rotation of knob 206 relative to body 208 results in a change in the longitudinal distance between knob 206 and body 208, with the distance increasing or decreasing depending on the direction of relative rotation. The change in longitudinal distance per unit of relative rotation of knob 206 and body 208 is related to the pitch of the meshing threads.

In one embodiment first spring 214 and second spring 216 are helical compression springs. In one embodiment first spring 214 and second spring 216 are helical compression springs with plain and ground ends. In one embodiment first spring 214 and second spring 216 are helical compression spring with squared and ground ends. In one embodiment first spring 214 and second spring 216 are compliant elastic members in the shape of a hollow cylinder or another geometry. In one embodiment first spring 214 and second spring 216 are identical such that they have the same dimensions, are made of the same material, and have the same stiffness properties. In one embodiment first spring 214 and second spring 216 are distinct such that they have different dimensions, or are made of different materials, or have different stiffness properties.

In one embodiment first pin 218 and second pin 220 are cylindrically shaped pins with their longitudinal axes oriented along the longitudinal axis of torquer 200. In one embodiment first pin 218 and second pin 220 are identical such that they have the same dimensions and are made of the same material. In one embodiment first pin 218 and second pin 220 have different dimensions or are made of different materials. In one embodiment the outer diameter of first pin 218 is equal to or larger than the outer diameter of first spring 214 and the outer diameter of second pin 220 is equal to or larger than the outer diameter of second spring 216.

Housing 222 includes a distal portion 222a, a first intermediate portion 222b, a second intermediate portion 222c, and a proximal portion 222d, with the longitudinal centerlines of all portions aligned with the longitudinal centerline of torquer 200. In one embodiment distal portion 222a of housing 222 is a support tube with a lumen 226 that extends distally to limit buckling and prevent kinking of a portion of the EMD along its length as the EMD is being translated and/or rotated. In one embodiment distal portion 222a of housing 222 is a cylindrical support tube with a lumen 226.

First intermediate portion 222b of housing 222 is a transition section integrally connecting distal portion 222a at its distal end and second intermediate portion 222c at its proximal end, and with lumen 226 extending throughout along its longitudinal centerline. In one embodiment first intermediate portion 222b comprises a double cone truncated at both apex ends with connected common cone bases with lumen 226 extending throughout along its longitudinal centerline. In one embodiment the diameters of the connected cone bases of the truncated double cone are identical and are smaller than the inner diameter of the distal portion 206a of knob 206. In one embodiment first intermediate portion 222b includes a distal truncated cone and a proximal truncated cone, where the distal truncated cone transitions from the outer diameter of distal portion 222a to the outer circular surface of second intermediate portion 222c. In one embodiment first intermediate portion 222b includes a distal truncated cone with a conical surface that increases in diameter in the longitudinal proximal direction 104 and a proximal truncated cone with a conical surface that decreases in the longitudinal proximal direction 104 and a flat proximal face. In one embodiment first intermediate portion 222b includes double cones with conical surfaces of arcuate profile.

Second intermediate portion 222c of housing 222 integrally connects first intermediate portion 222b and proximal portion 222d, with first intermediate portion 222b at its distal end and with proximal portion 222d at its proximal end. In one embodiment second intermediate portion 222c is of cylindrical-shape and includes a first pocket 222e and a second pocket 222f, both recessed within second intermediate portion 222c and both oriented along the longitudinal axis of second intermediate portion 222c, and includes a conical portion 222g at its proximal end. In one embodiment the length of first pocket 222e is larger than the length of first jaw 210 and the length of second pocket 222f is larger than the length of second jaw 212. In one embodiment the width of first pocket 222e is larger than the width of first jaw 210 and the width of second pocket 222f is larger than the width of second jaw 212. In one embodiment the base of conical portion 222g is at the proximal end of second intermediate portion 222c of housing 222 and the diameter of the conical surface is decreasing in the longitudinal distal direction 202.

In one embodiment proximal portion 222d includes a distal portion 222h and a proximal portion 222i, where distal portion 222h is a circular cylinder, such as a disk, with an outer diameter and an inner diameter and where proximal portion 222i is a bevel gear with its teeth facing the proximal direction. In one embodiment the distal portion 222h and the proximal portion 222i of proximal portion 222d are a single manufactured component, such as a molded component, with an internal pathway through which passes a portion of the shaft of the EMD. In one embodiment the distal portion 222h and the proximal portion 222i of proximal portion 222d are affixed together as one integrated unit, with an internal pathway through which passes a portion of the shaft of the EMD. In one embodiment proximal portion 222d of housing 222 includes two holes that extend longitudinally throughout, where the diameters of the holes are larger than the diameters of the threaded portion of fasteners 224, and where the holes are located toward the periphery of proximal portion 222d and match the locations of recessed threads of the proximal portion 208c of body 208. In one embodiment bevel gear is a driven member operatively driven by a drive member in a robotic system.

In one embodiment housing 222 is a single manufactured component, such as a molded component, with an internal pathway through which passes a portion of the shaft of the EMD. In one embodiment housing 222 is an assembled component, with an internal pathway through which passes a portion of the shaft of the EMD.

In one embodiment of assembled torquer 200 body 208 is removably fixed to housing 222 by means of fasteners 224 that are inserted in the holes of the proximal portion 222d of housing 222 and threaded into recessed threaded holes of the proximal portion 208c of body 208. In one embodiment of assembled torquer 200 body 208 is fixed to housing 222 by means of glue, adhesive, bonding agent, laser welding, ultrasonic welding, or other means of affixing two bodies during assembly and manufacture.

In one embodiment of torquer 200 first spring 214 is inserted in the longitudinal distal direction 202 and fully located within hole 210h of first jaw 210 and second spring 216 is inserted in the longitudinal distal direction 202 and fully located within a similar hole of second jaw 212. In one embodiment of torquer 200 the distal end of first spring 214 is pressed against the distal end of hole 210h of first jaw 210 and the distal end of second spring 216 is pressed against the distal end of a similar hole of second jaw 212. In one embodiment of torquer 200 first pin 218 is inserted in the longitudinal distal direction 202 and its distal end contacts the proximal end of first spring 214 within hole 210h of first jaw 210 and second pin 220 is inserted in the longitudinal distal direction 202 and its distal end contacts the proximal end of second spring 216 within a similar hole of second jaw 212.

In one embodiment of torquer 200 first pad base 210b is kinematically constrained in first pocket 222e of housing 222 and second pad base 212b is kinematically constrained in second pocket 222f of housing 222. In particular, in one embodiment the walls of first pocket 222e constrain lateral motion of first jaw 210 (by contacting flat front lateral surface 210d and flat back lateral surface 210e of first pad base 210b) and the walls of second pocket 222f constrain lateral motion of second jaw 212.

In one embodiment a portion of the top surface of first pad base 210b of first jaw 210 contacts a portion of the inner circumferential wall of body 208 and a portion of the bottom surface of second pad base 212b of second jaw 212 contacts a portion of the inner circumferential wall of body 208. In one embodiment a portion of the top surface of first pad base 210b of first jaw 210 contacts a portion of the inner circumferential wall of body 208 and a portion of the top surface of first pad base 210b of first jaw 210 contacts a portion of the inner circumferential wall of housing 222, and a portion of the bottom surface of second pad base 212b of second jaw 212 contacts a portion of the inner circumferential wall of body 208 and a portion of the bottom surface of second pad base 212b of second jaw 212 contacts a portion of the housing 222.

In one embodiment torquer 200 includes two jaws that move relative to each other to releasably fix a portion of the shaft of the EMD to at least one of the two jaws. In one embodiment torquer 200 includes one jaw that moves relative to the body of the torquer 200 to releasably fix a portion of the shaft of the EMD to the one jaw. In one embodiment torquer 200 includes more than two jaws that move relative to each other to releasably fix a portion of the shaft of the EMD to at least one of the jaws.

In one embodiment of torquer 200 first spring 214 acts as a biasing member that biases one jaw relative to the body. In one embodiment of torquer 100 first spring 214 and second spring 216 act as biasing members that bias two jaws relative to the body. In one embodiment of torquer 100 more than two springs act as biasing members that bias more than two jaws relative to the body.

Referring to FIGS. 15, 16, and 17 active torquer 200 in accordance with an embodiment is shown in stages corresponding to a fully unpinched state, an unpinched state in transition to pinched state, and a pinched state, respectively. In an unpinched state torquer 200 is in a disengaged position and is not pinching EMD 228, and in a pinched state torquer 200 is in a fully engaged position and is pinching a portion of EMD 228. In the embodiment depicted in all three states (unpinched, unpinched in transition to pinched, and pinched) the internal threads 206c of knob 206 mesh with the external threads 208d of body 208.

Referring to FIG. 15 in the fully unpinched state of torquer 200 knob 206 is in an open position relative to body 208. In the unpinched state of torquer 200 EMD 228 in lumen 226 can be withdrawn in a longitudinal proximal direction 204 or EMD 228 can be inserted in lumen 226 in a longitudinal distal direction 202. There is no contact (that is, a gap exists) between the third intermediate portion 210m of the top surface of the first pad base 210b of first jaw 210 and the sloped inner wall of the proximal portion 208c of body 208, and there is no contact between the corresponding intermediate portion of the bottom surface of the second pad base 212b of second jaw 212 and the sloped inner wall of the proximal portion 208c of body 208. Rotating knob 206 relative to body 208 in a direction that unthreads knob 206 from body 208 causes knob 206 to move in a longitudinal distal direction 202 relative to body 208 increasing the gap between the third intermediate portion 210m of the top surface of the first pad base 210b of first jaw 210 and the sloped inner wall of the proximal portion 208c of body 208, and between the corresponding intermediate portion of the bottom surface of the second pad base 212b of second jaw 212 and the sloped inner wall of the proximal portion 208c of body 208.

In one embodiment knob 206 freely rotates relative to body 208 in a direction that unthreads knob 206 from body 208 until their teeth no longer mesh and knob 206 separates from body 208. In one embodiment knob 206 freely rotates relative to body 208 in a direction that unthreads knob 206 from body 208 until a stop is reached preventing knob 206 from separating from body 208.

In the fully unpinched state of torquer 200 in which knob 206 is in an open position relative to body 208 there is contact between the proximal conical surface of the first intermediate portion 222b of housing 222 and the inclined distal surface 210f of first pad base 210b of first jaw 210, and there is contact between the proximal conical surface of the first intermediate portion 222b of housing 222 and the inclined distal surface of second pad base 212b of second jaw 212.

In the fully unpinched state of torquer 200 first pad 210a of first jaw 210 and second pad 212a of second jaw 212 face each other, are separated from one another by a distance, are parallel to each other and to a portion of EMD 228, if present, and not in contact with any portion of EMD 228, that is, first pad 210a and second pad 212a are not in contact with a portion of EMD 228 over the length of each pad.

In the fully unpinched state of torquer 200 first spring 214 and second spring 216 are compressed relative to their respective rest lengths. As a result, a spring restoring force acts in the longitudinal distal direction 202 from first spring 214 and a spring restoring force acts in the longitudinal distal direction 202 from second spring 216. (Spring restoring forces also act in the longitudinal proximal direction 204 from first spring 214 and from second spring 216 for static equilibrium. However, the proximal end of first spring 214 and the proximal end of second spring 216 are constrained, that is, fixed relative to housing 222 and to body 208 to which housing 222 is affixed. The lengths of first pin 218 and second pin 220 are constant and both pins are constrained from moving relative to housing 222 and to body 208 as their proximal ends contact the conical portion 222g of second intermediate portion 222c of housing 222. Thus, the spring restoring forces of interest act in the longitudinal distal direction 202.)

The spring restoring force from first spring 214 acts on first jaw 210 by contact between the distal end of first spring 214 and the internal surface at the distal end of hole 210h of first pad base 210b. As a result, first jaw 210 moves in the longitudinal distal direction 202 until it is constrained from motion by contact between the first intermediate portion 210j of the top surface of first pad base 210b and the inclined internal wall of knob 206. Similarly, the spring restoring force from second spring 216 acts on second jaw 212 by contact between the distal end of second spring 216 and the internal surface at the distal end of the hole of second pad base 212b. As a result, second jaw 212 moves in the longitudinal distal direction 202 until it is constrained from motion by contact between the corresponding first intermediate portion of the bottom surface of second pad base 212b and the inclined internal wall of knob 206.

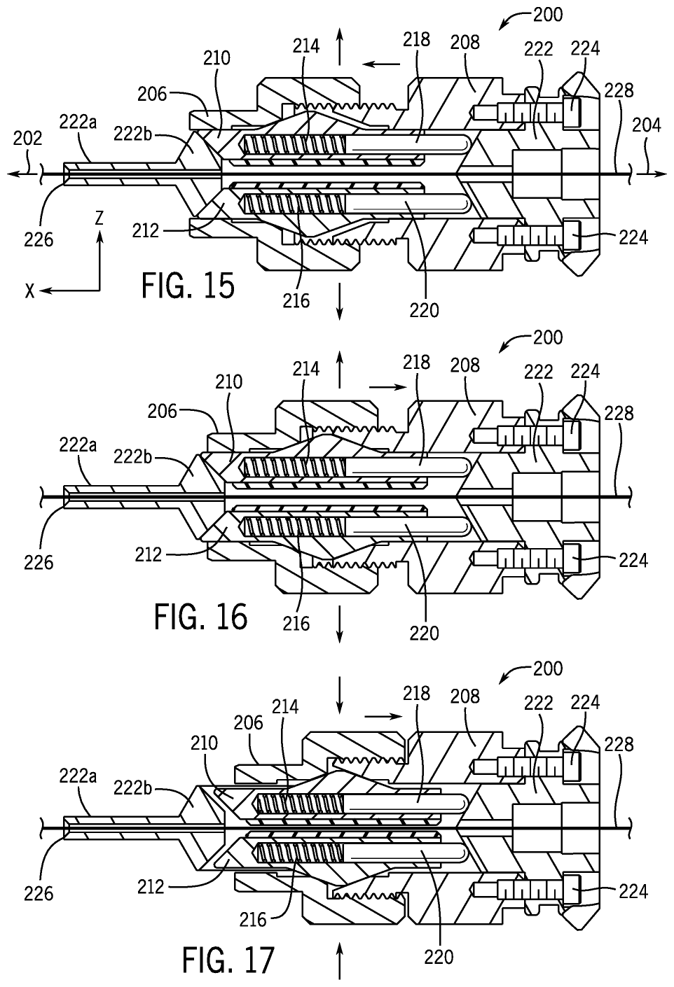

Referring to FIG. 16 in the unpinched state of torquer 200 in the transition from fully unpinched to fully pinched state knob 206 is in a partially closed position relative to body 208. Rotating knob 206 relative to body 208 in a direction that threads knob 206 toward body 208 causes knob 206 to move in a longitudinal proximal direction 204 relative to body 208 such that there is contact between the third intermediate portion 210m of the top surface of the first pad base 210b of first jaw 210 and the sloped inner wall of the proximal portion 208c of body 208, and there is contact between the corresponding intermediate portion of the bottom surface of the second pad base 212b of second jaw 212 and the sloped inner wall of the proximal portion 208c of body 208.

As knob 206 is threaded toward body 208 causing knob 206 to move in a longitudinal proximal direction 204 relative to body 208 it pushes first jaw 210 and second jaw 212 in the longitudinal proximal direction 204. Motion in the longitudinal proximal direction 204 compresses first spring 214 further than it is compressed in the unpinched state giving rise to a larger magnitude of the first spring restoring force than the magnitude of the first spring restoring force in the unpinched state, and compresses second spring 216 further than it is compressed in the unpinched state giving rise to a larger magnitude of the second spring restoring force than the magnitude of the second spring restoring force in the unpinched state.

Due to the sloped inner wall of knob 206, knob 206 also pushes first jaw 210 down toward the longitudinal central axis of torquer 200 and pushes second jaw 212 up toward the longitudinal central axis of torquer 200. In other words, first pad 210a of first jaw 210 moves radially toward a portion of EMD 228 and second pad 212a of second jaw 212 moves radially toward a portion of EMD 228. The first pad 210a and the second pad 212a remain in a parallel orientation to each other and to a portion of EMD 228 between them as a result of the two inclined surfaces of the upper surface of first pad base 210b and the two inclined surfaces of the lower surface of second pad base 212b. In particular, the slope of the first intermediate portion 210j of the top surface of first pad base 210b is the same as the slope of the internal wall of knob 206 and the slope of the third intermediate portion 210m of the top surface of first pad base 210b is the same as the slope of the internal wall of body 208 the flat bottom lower surface 210c of first pad base 210b. In addition, the slope of the first intermediate portion 210j of the top surface of first pad base 210b has the same magnitude and opposite sign as the slope of the third intermediate portion 210m of the top surface of first pad base 210b. As a result, the flat bottom lower surface 210c of first pad base 210b as well as the first pad 210a remain parallel to the longitudinal central axis of torquer 200 and move toward the longitudinal central axis of torquer 200 as knob 206 is threaded toward body 208. Similarly, the flat top upper surface of second pad base 212b as well as the second pad 212a remain parallel to the longitudinal central axis of torquer 200 and move toward the longitudinal central axis of torquer 200 as knob 206 is threaded toward body 208.

Referring to FIG. 17 in the pinched state of torquer 200 knob 206 is in a closed position relative to body 208. In the pinched state of torquer 200 first pad 210a of first jaw 210 and second pad 212a of second jaw 212 face each other, are parallel to each other and to a portion of EMD 228, and pinch a portion of EMD 228 over the length of each pad, that is, first pad 210a and second pad 212a are in contact with a portion of EMD 228 over the length of each pad. Fully rotating knob 206 relative to body 208 in a direction that threads knob 206 toward body 208 until no further travel is possible causes knob 206 to move to its most proximal position relative to body 208.

With knob 206 in its most proximal position relative to body 208 knob 206 pushes first jaw 210 and second jaw 212 in the longitudinal proximal direction 204 to their most proximal achievable positions, corresponding to maximum compression of first spring 214 and maximum compression of second spring 216. A maximum restoring force from first spring 214 develops and acts in the longitudinal distal direction 202 on first jaw 210 and a maximum restoring force from second spring 216 develops and acts in the longitudinal distal direction 202 on second jaw 212. Thus, a maximum vertical force component acts in pressing first jaw radially down toward the longitudinal central axis of torquer 200 and a maximum vertical force component acts in pressing first jaw radially up toward the longitudinal central axis of torquer 200.

As first pad 210a and second pad 212a are pressed toward one another, and each into EMD 228 with a circular cross-section, first pad 210a and second pad 212a each deform slightly around EMD 228 and there is contact between the bottom surface of first pad 210a and a portion of the circumference of EMD 228 over the length of first pad 210a and there is contact between the top surface of second pad 212a and a portion of the circumference of EMD 120 over the length of second pad 212a.

As the first pad 210a deforms partially around EMD 228 and second pad 212a deforms partially around EMD 228, that is, each conforming to an arc of the circular cross-section of EMD 228 over their lengths, they are pressed toward one another from opposite directions and EMD 228 is pinched between them.

In one embodiment the pad with a compliant property has a magnitude of a modulus between 200 to 400 MPa. In one embodiment the pad with a compliant property has a durometer value of the pads is between 45D to 75D. In one embodiment the pad with a compliant property has a magnitude of a modulus between 200 to 400 MPa and durometer value of the pads is between 45D to 75D. In one embodiment the force applied to the EMD from the jaw pads has a magnitude between 200 to 400 N. In one embodiment the length of the pads is less than or equal to 50 mm. In one embodiment the modulus of the pad base has a value equal to or greater than 3.5 GPa. In one embodiment the modulus of the pad base is greater than the modulus of the pads. In one embodiment the modulus of the elastomeric pads has a value between 200 to 400 MPa, the length of the elastomeric pads has a value equal to or less than 50 mm, the durometer of the elastomeric pads has a value between 45D to 75D, and the modulus of the pad base has a value equal to or greater than 3.5 GPa. The jaw pads referred to in this paragraph are the jaw pads in torquer 100 and torquer 200.

As described herein in the engaged state of torquer 200 the EMD shaft is pinched in the internal pathway of torquer 200. In one embodiment the EMD is axially loaded into the internal pathway of torquer 200. In axial loading a shaft portion is loaded into the internal pathway of torquer 200 by first inserting a free end of the EMD into a proximal or distal opening of lumen 226.

In another embodiment the EMD shaft is radially loaded into the internal pathway of the torquer. Radially loaded contrasts with axially loaded and may also be referred to as side-loaded or laterally loaded. The EMD is loaded into the torquer 200 through a longitudinal slit or opening of the torquer (that is the side of the torquer extending from a proximal end to the distal end of the torquer). In a radially loaded embodiment access to the internal pathway occurs by means of a longitudinal slit along torquer 200 from its outer periphery to the internal pathway.

Referring to FIG. 18 torquer 100 is positioned in one embodiment of device module 32. Although not shown torquer 200 can also be positioned within a device module to robotically control an EMD. Cassette 66 of device module 32 includes a bearing support 232 that receives a bearing surface 117 on torquer 100. Device module bearing support 232 provides rotational and thrust support of torquer 100 such that torquer 100 can rotate about the longitudinal axis of the device module while the device module itself does not rotate. Distal portion 106a of knob 106 provides anti-buckling support for the EMD. Bearing support 232 in one embodiment is formed by a C shaped bracket in cassette 66 and in one embodiment bearing support 232 is formed partially by a bracket within cassette 66 and partially by a portion of a cover pivotally attached to cassette 66.

In one embodiment the distal free end of the distal portion 106a is closely adjacent to the device support or flexible track 79 along the longitudinal axis of the device module such that the EMD does not buckle between the distal end of the distal portion 106a and the track 79 when the EMD is being translated and/or rotated. In one embodiment the distance between the distal free end of distal portion 106a and the device support or track 79 is less than one inch (25.4 mm) and in one embodiment less than 0.5 inches (12.7 mm). In one embodiment the distal free end of the distal portion 106a is located within the lumen defined by the device support or track 79. In one embodiment track 79 is formed from a flexible member that moves from a position co-linear to the longitudinal axis of the device module to a position off set from the longitudinal axis of the device. In one embodiment track 79 has a longitudinal slit extending from an outer surface of track 79 to a lumen extending longitudinally therethrough. In the in use position driven member 116b engages a drive member 230. The drive member is robotically controlled to impart rotational movement to the torquer and EMD.

The distal end of distal portion 106a in one embodiment In one embodiment a torquer for use with certain EMDs such as a stent retriever and certain coils where it is undesirable to rotate the proximal shaft, the adaptor is not provided with a driven member. In one embodiment the adaptor includes a feature such as a tab that engages with a stop on the cassette or device module to prevent rotation of the adaptor and certain EMDs.

Passive torquer 100 resembles a two jawed collet with a pre-loaded spring that pinches the elastomer pads together. When the operator screws the knob in, the jaws are forced into an open position. To close the jaws again, the knob is unscrewed, releasing the jaws. Passive torquer does not damage EMDs, accepts the full range of wire-like device sizes, and eliminates performance variance due user strength. Variations of the invention include axial vs. radial loading, single side pinching, alternate force sources, and alternate actuation mechanisms.

The use of elastomeric materials on the pads minimizes damage to the outer surface coating of EMDs as compared to jaws being formed from a metal material.

While other types of torque devices are available, the most commonly used torque devices require the operator to tighten a knob to close a pin vise on the wire like device. The rotational and linear grip of the torquer is dependent on how tightly the operator tightens the knob. Human strength testing suggests that more that 5% of the population will not be able to adequately torque the knob to achieve the target torque performance of 2.5 mNm guidewire torque. Significantly fewer people will be able to provide enough knob torque to achieve 6.5 mNm torque on an access wire having a diameter range of 0.035-0.038 inch (0.889-0.965 mm) diameter device.

Although the present disclosure has been described with reference to example embodiments, workers skilled in the art will recognize that changes may be made in form and detail without departing from the spirit and scope of the defined subject matter. For example, although different example embodiments may have been described as including one or more features providing one or more benefits, it is contemplated that the described features may be interchanged with one another or alternatively be combined with one another in the described example embodiments or in other alternative embodiments. Because the technology of the present disclosure is relatively complex, not all changes in the technology are foreseeable. The present disclosure described is manifestly intended to be as broad as possible. For example, unless specifically otherwise noted, the definitions reciting a single particular element also encompass a plurality of such particular elements.

What is claimed is:

1. A torquer for an elongated medical device:
a body having a cavity defining a pathway;
a first jaw movable within the cavity, the first jaw including a first pad having a compliant property;
a biasing member separate from the first jaw biasing the first jaw relative to the body; and
an actuator movable relative to the body moving the first jaw pinching and/or unpinching the elongated medical device with the first pad within the pathway.

2. The torquer of claim 1, wherein the first pad is formed from an elastomeric material and further including at least a second jaw movable relative to the body toward the first jaw, the second jaw having a second pad formed from an elastomeric material, wherein each jaw includes a pad base having a first modulus that is greater than a second modulus of the first and second pads.

3. The torquer of claim 2, wherein each jaw is free to pivot about a cam surface on the body independent of one another and wherein the jaws are not connected to one another.

4. The torquer of claim 3, wherein a distal end and a proximal end of the elastomeric pad move radially away from a torquer longitudinal axis as the jaw pivots about the cam surface.

5. The torquer of claim 2, wherein the body includes a cam surface contacting a nonlinear follower surface on each pad base.

6. The torquer of claim 5, wherein the follower surface is arcuate.

7. The torquer of claim 6, wherein the cam surface is linear.

8. The torquer of claim 1, wherein the pads are elastomeric pads and wherein pressure between the elastomeric pads and the elongated medical device is substantially equalized along an entire length that the elastomeric pads contact the elongated medical device in a fully pinched position.

9. The torquer of claim 2, wherein the biasing member biases the pads toward one another.

10. The torquer of claim 2, wherein the biasing member biases the pads away from one another.

11. The torquer of claim 1, wherein the biasing member includes one or more helical compression springs having a longitudinal axis being parallel or co-linear with a longitudinal axis of the body.

12. The torquer of claim 2, wherein each jaw has a distal end and a proximal end, wherein one of the distal ends and proximal ends of the jaws move away from one another prior to the other of the distal ends and proximal ends of the jaws.

13. The torquer of claim 1, wherein the pathway can an accommodate elongated medical device having a diameter of 0.014 inches through and including 0.038 inches.

14. The torquer of claim 2, wherein a magnitude of a force applied to the elongated medical device from the pads is between 200 to 400 N, and a magnitude of a modulus of the pads is between 200 to 400 MPa, and a length of the pads is less than 50 mm, and a durometer value of the pads is between 45D to 75D, and where a magnitude of a modulus of each pad base is greater than 3.5 GPa.

15. A torquer releasably pinching an elongated medical device, comprising:
a body having a cavity defining a pathway;
at least two jaws movable within the cavity, each jaw having a pad base and a pad secured thereto, wherein the jaws are not connected to one another;
a biasing member separate from the jaws biasing the jaws relative to the body; and
a knob movable relative to the body moving the jaws relative to one another pinching or unpinching the elongated medical device with the pads within the pathway.

16. The torquer of claim 15, wherein the body includes a cam surface contacting a nonlinear follower surface on each pad base.

17. The torquer of claim 16, wherein each jaw is free to pivot about the cam surface independent of one another.

18. The torquer of claim 17, wherein pressure between the pads and the elongated medical device is substantially equalized along an entire length of the pads.

19. The torquer of claim 15, wherein a magnitude of a force applied to the elongated medical device from the pads is between 200 to 400 N, and a magnitude of a modulus of the pads is between 200 to 400 MPa, and a length of the pads is less than 50 mm, and a durometer value of the pads is between 45D to 75D, and where a magnitude of a modulus of the pad base is greater than 3.5 GPa.

20. A torquer releasably engaging an elongated medical device, comprising:
a body having a cavity defining a pathway;
at least two jaws movable within the cavity, each jaw having an elastomeric pad, wherein the jaws are not connected to one another;
a biasing member separate from the jaws biasing the jaws relative to the body; and
a knob movable relative to the body moving the jaws relative to one another pinching or unpinching the elongated medical device with the elastomeric pads within the pathway;
wherein pressure between the elastomeric pads and the elongated medical device is substantially equalized along an entire length of the elastomeric pads in a fully pinched position.
